SEQUENCE LISTING (37 sequences)

SEQ ID NO: 1 - protein, 8 aa, Artificial Sequence
  Description of Artificial Sequence Synthetic peptide
AQGVLPGQ

SEQ ID NO: 2 - protein, 8 aa, Artificial Sequence
  Description of Artificial Sequence Synthetic peptide
LQGVLPGQ

SEQ ID NO: 3 - protein, 8 aa, Artificial Sequence
  Description of Artificial Sequence Synthetic peptide
AQGLQPGQ

SEQ ID NO: 4 - protein, 8 aa, Artificial Sequence
  Description of Artificial Sequence Synthetic peptide
LQGLQPGQ

SEQ ID NO: 5 - protein, 4 aa, Artificial Sequence
  Description of Artificial Sequence Synthetic peptide
AQGV

SEQ ID NO: 6 - protein, 5 aa, Artificial Sequence
  Description of Artificial Sequence Synthetic peptide
LQGVL

SEQ ID NO: 7 - protein, 5 aa, Artificial Sequence
  Description of Artificial Sequence Synthetic peptide
AQGLQ

SEQ ID NO: 8 - protein, 5 aa, Artificial Sequence
  Description of Artificial Sequence Synthetic peptide
LQGLQ

SEQ ID NO: 9 - protein, 4 aa, Artificial Sequence
  Description of Artificial Sequence Synthetic peptide
LQGV

SEQ ID NO: 10 - protein, 6 aa, Artificial Sequence
  Description of Artificial Sequence Synthetic peptide
VLPALP

SEQ ID NO: 11 - protein, 5 aa, Artificial Sequence
  Description of Artificial Sequence Synthetic peptide
RIVPA

SEQ ID NO: 12 - protein, 12 aa, Artificial Sequence
  Description of Artificial Sequence Synthetic peptide
VRLIVAVRIWRR

SEQ ID NO: 13 - protein, 7 aa, Artificial Sequence
  Description of Artificial Sequence Synthetic peptide
AQLPGVI

SEQ ID NO: 14 - protein, 6 aa, Artificial Sequence
  Description of Artificial Sequence Synthetic peptide
AQLPVG

SEQ ID NO: 15 - protein, 4 aa, Artificial Sequence
  Description of Artificial Sequence Synthetic peptide
AQLP

SEQ ID NO: 16 - protein, 4 aa, Artificial Sequence
  Description of Artificial Sequence Synthetic peptide
LIVA

SEQ ID NO: 17 - protein, 4 aa, Artificial Sequence
  Description of Artificial Sequence Synthetic peptide
PIVA

SEQ ID NO: 18 - protein, 4 aa, Artificial Sequence
  Description of Artificial Sequence Synthetic peptide
QQGV

SEQ ID NO: 19 - protein, 4 aa, Artificial Sequence
  Description of Artificial Sequence Synthetic peptide
GQGV

SEQ ID NO: 20 - protein, 4 aa, Artificial Sequence
  Description of Artificial Sequence Synthetic peptide
PQGV

SEQ ID NO: 21 - protein, 4 aa, Artificial Sequence
  Description of Artificial Sequence Synthetic peptide
VQGV

SEQ ID NO: 22 - protein, 4 aa, Artificial Sequence
  Description of Artificial Sequence Synthetic peptide
ALGV

SEQ ID NO: 23 - protein, 4 aa, Artificial Sequence
  Description of Artificial Sequence Synthetic peptide
LLGV

SEQ ID NO: 24 - protein, 4 aa, Artificial Sequence
  Description of Artificial Sequence Synthetic peptide
QLGV

SEQ ID NO: 25 - protein, 4 aa, Artificial Sequence
  Description of Artificial Sequence Synthetic peptide
GLGV

SEQ ID NO: 26 - protein, 4 aa, Artificial Sequence
  Description of Artificial Sequence Synthetic peptide
PLGV

SEQ ID NO: 27 - protein, 4 aa, Artificial Sequence
  Description of Artificial Sequence Synthetic peptide
VLGV

SEQ ID NO: 28 - protein, 4 aa, Artificial Sequence
  Description of Artificial Sequence Synthetic peptide
QAGV

SEQ ID NO: 29 - protein, 4 aa, Artificial Sequence
  Description of Artificial Sequence Synthetic peptide
QGGV

SEQ ID NO: 30 - protein, 4 aa, Artificial Sequence
  Description of Artificial Sequence Synthetic peptide
QPGV

SEQ ID NO: 31 - protein, 4 aa, Artificial Sequence
  Description of Artificial Sequence Synthetic peptide
QVGV

SEQ ID NO: 32 - protein, 4 aa, Artificial Sequence
  Description of Artificial Sequence Synthetic peptide
LAGV

SEQ ID NO: 33 - protein, 4 aa, Artificial Sequence
  Description of Artificial Sequence Synthetic peptide
LGGV

SEQ ID NO: 34 - protein, 4 aa, Artificial Sequence
  Description of Artificial Sequence Synthetic peptide
LPGV

SEQ ID NO: 35 - protein, 4 aa, Artificial Sequence
  Description of Artificial Sequence Synthetic peptide
LVGV

SEQ ID NO: 36 - protein, 7 aa, Artificial Sequence
  Description of Artificial Sequence Synthetic peptide
AQGVLPG

SEQ ID NO: 37 - protein, 6 aa, Artificial Sequence
  Description of Artificial Sequence Synthetic peptide
AQGVLP

(19) United States
(12) Patent Application Publication
(10) Pub. No.: US 2024/0016882 A1
(43) Pub. Date: Jan. 18, 2024

(54) AUTOPHAGY-INHIBITING PEPTIDE AND ORGANIC ACID SALT THEREOF ADDRESSING ISSUES OF VASCULAR PERMEABILITY

(71) Applicant: BIOTEMPT B.V., Gravenhage (NL)

(72) Inventors: Gert WENSVOORT, Koekange (NL); Johan RENES, Amsterdam (NL)

(73) Assignee: BIOTEMPT B.V., Gravenhage (NL)

(21) Appl. No.: 18/029,174

(22) PCT Filed: Sep. 29, 2021
(86) PCT No.: PCT/EP2021/076844

§ 371 Date: Mar. 29, 2023

Related U.S. Application Data
(60) Provisional application No. 63/085,771, filed on Sep. 30, 2020.

(51) Int. Cl. A61K 38/08

(52) U.S. Cl. CPC A61K 38/08

(57) ABSTRACT

The invention relates generally to biotechnology and medicine and to sources and salts of autophagy inhibiting peptides useful as pharmaceutical compounds. In particular, the invention provides method for reducing formyl-peptide-receptor (FPR) mediated p38 MAPK kinase activity of cells comprising providing said cells with a source of amino acids said source comprising at least 50%, more preferably at least 75%, most preferably at 100% amino acids selected from the group of autophagy inhibiting amino acids alanine (in one letter code: A), glutamine (Q), glycine (G), valine (V), leucine (L), proline (P), and arginine (R).

### CROSS REFERENCE TO RELATED APPLICATIONS

The present application is a § 371 National Phase application based on PCT/EP2021/076844, filed Sep. 29, 2021, which claims the benefit of U.S. provisional application No. 63/085,771, filed Sep. 30, 2020, the subject matter of each of which is incorporated by reference in their entirety.

### SEQUENCE LISTING

The instant application contains a Sequence Listing which has been submitted electronically in ASCII format and is hereby incorporated by reference in its entirety. Said ASCII copy, created on Sep. 26, 2023, is named 66911-2_SL.txt and is 7,563 bytes in size.

### FIELD OF THE INVENTION

The invention relates generally to biotechnology and medicine and to pharmaceutical compounds, aqueous solutions and salts useful as pharmaceutical compounds, capable of modulating vascular barrier function, in particular capable of modulating adverse vascular permeability that may result in edema, adverse vascular leakage, adverse leukocyte extravasation and hypotension. The invention provides pharmaceutical formulations, solutions, methods, and means to achieve such solutions to treat issues of adverse vascular permeability that for example is frequently found in critically ill patients.

### BACKGROUND

In critically ill patients, in order to restore cardiac output, systemic blood pressure and renal perfusion with an adequate fluid resuscitation is essential. Achieving an appropriate level of volume management requires knowledge of the underlying pathophysiology, evaluation of volume status, and selection of appropriate solution for volume repletion, and maintenance and modulation of the tissue perfusion. Numerous recent studies have established a correlation between fluid overload (also indicated as positive fluid balance) and mortality in critically ill patients. Fluid overload recognition and assessment requires an accurate documentation of intakes and outputs; yet, there is a wide difference in how it is evaluated, reviewed and utilized. Accurate volume status evaluation is essential for appropriate therapy since errors of volume evaluation can result in either in lack of essential treatment or unnecessary fluid administration, and both scenarios are associated with increased mortality. There are several methods to evaluate fluid status; however, most of the tests currently used are fairly inaccurate. Diuretics, especially loop diuretics, remain a valid therapeutic alternative. Fluid overload refractory to medical therapy requires the application of extracorporeal therapies. Fluid overload is typically related to increased mortality and may also lead to several complications like pulmonary oedema, cardiac failure, delayed wound healing, tissue breakdown, and impaired bowel function. Therefore, the evaluation of volume status is crucial in the early management of critically ill patients and probably even more important to patients at risk of becoming critically ill. Diuretics are frequently used as an initial therapy; however, due to their limited effectiveness the use of continuous renal replacement techniques are often required for fluid overload treatment. Successful fluid overload treatment depends on precise assessment of individual volume status, understanding the principles of fluid management with ultrafiltration, and clear treatment goals. In addition, an improved mechanistic understanding of potential harm from excessive aqueous fluid administration (Marik P E. Iatrogenic salt water drowning and the hazards of a high central venous pressure. Ann Intensive Care. 2014 Jun. 21; 4:21) and emerging observational data associating positive fluid balance with higher mortality (Sakr et al; Higher Fluid Balance Increases the Risk of Death From Sepsis: Results From a Large International Audit. Crit Care Med. 2017 March; 45(3):386-394.) have recently challenged the paradigm of large-volume fluid resuscitation.

Thus, although prompt intravenous fluid therapy is a fundamental treatment for patients, an optimal approach for administering intravenous fluid in resuscitation is unknown, and moreover complicated by the vasopressors one often uses to issues of vascular permeability of a patient. Two competing resuscitation strategies are emerging—a liberal fluids approach consisting of a larger volume of initial fluid [50-75 ml/kg (4-6 litres in an 80 kg adult) over the first 6 hours] and later use of vasopressors, versus a restrictive fluids approach consisting of a smaller volume of initial fluid [30 ml/kg (2-3 litres)] with earlier reliance on vasopressor infusions to maintain blood pressure and perfusion. Early fluid therapy may enhance or maintain tissue perfusion by increasing venous return and cardiac output. However, fluid administration may also have deleterious effects by causing oedema within vital organs, leading to organ dysfunction and impairment of oxygen delivery. Conversely, a restrictive fluids approach primarily relies on vasopressors to reverse hypotension and maintain perfusion while limiting the administration of fluid. Both strategies have some evidence to support their use, but lack robust data to confirm the benefit of one strategy over the other, creating a clinical and scientific equipoise. Furthermore, several types of resuscitation fluids exist. Two main classes are differentiated, crystalloids and colloids. Crystalloids typically have small molecules, are cheap, easy to use, and provide immediate fluid resuscitation, but may increase oedema. Colloids have larger molecules, cost more, and may provide swifter volume expansion in the intravascular space, but may induce allergic reactions, blood clotting disorders, and kidney failure.

The vasculature, composed of vessels of different morphology and function, distributes blood to all tissues and maintains physiological tissue homeostasis. Among others, to illustrate its central role in maintaining homeostasis, the vasculature not only serves as the main carrier in gas exchange from lung to tissues (in particular oxygen (and vice versa in particular carbon dioxide)) but also carries nutrients from gut to liver to tissues and toxic by-products resulting metabolism from tissues to kidney to urine for excretion.

Whereas vascular permeability is an important functionality in healthy humans, in a range of pathologies the vasculature is often affected by, and involved in, the disease process. This primarily results in adverse vascular permeability with edema, adverse vascular leakage, adverse leukocyte extravasation and hypotension and may also result in excessive formation of new, unstable, and hyper permeable vessels with poor blood flow, which further promotes hypoxia and disease propagation. Chronic adverse vessel permeability also facilitates metastatic spread of cancer. Thus, there is a strong incentive to learn more about (and be able to modulate) an important aspect of vessel biology in health and disease: the regulation of vessel permeability.

Endothelial cells in different vessels and in different organs have distinct functions and morphologies (Aird W C. Molecular heterogeneity of tumor endothelium. Cell Tissue Res. 2009; 335:271-81.), but in general serve to provide a barrier between blood and tissue. In certain organs, such as the brain and in endocrine organs, endothelial cells present certain morphological features that reflect the need for communication between the organs and the circulation. In the brain, the vasculature forms a particularly strong barrier, the blood-brain barrier (BBB) to protect the brain parenchyma from detrimental edema. In hormone-producing organs, such as the endocrine pancreas, endothelial cells display specialized fenestrae on their surface. These are diaphragm-covered ‘holes’ in the plasma membrane, which allow extremely rapid exocytosis of hormones. In most organs, the endothelial cells form a dynamic barrier between the blood and the tissue. In resting conditions, the vasculature continuously leaks solute and small molecules but restricts extravasation of larger molecules and cells. In many diseases, including cancer, the vascular barrier disintegrates and leakage increases and may become chronic. The leakage of larger molecules and cells may result in edema, adverse leukocyte extravasation and hypotension, and often disease progression.

It is well recognized that for example kinins such as bradykinin are involved in a series of physiological and sometimes pathological vascular responses affecting endothelial barrier function. Most of their actions are mediated by the activation of 2 G protein-coupled receptors, named B₁ and B₂. The activation of kinin receptors may play a key role in the modulation of atherosclerotic risk through the promotion of microangiogenesis, inhibition of vascular smooth muscle cell growth, coronary vasodilatation, increased local nitric oxide synthesis, or by exerting antithrombotic actions. The bradykinin B₁ receptor (B₁R) is typically absent under physiological conditions, but is highly inducible following tissue injury, stress, burns, traumatic damage, such as for example recently reported in COVID-19 disease.

Damage induced by tissue injury may cause a significant and time-dependent increase in des-Arg⁹-bradykinin (des-Arg⁹-BK) responsiveness that parallels B₁R mRNA expression. It induces the activation of some members of the mitogen activated protein kinase (MAPK) family, namely, extracellular signal-regulated kinase (ERK) and p38 MAPK. The blockade of p38 MAPK but not ERK pathways with selective inhibitors, results in a significant reduction of the upregulated contractile response caused by the selective B₁R agonist des-Arg⁹-BK, and largely prevents the induction of B₁R mRNA expression enhancing tissue damage induced adverse vascular permeability.

Among other stress stimuli, exposure to hypoxia as a consequence of impaired blood flow, or as a consequence of impaired gas exchange between alveoli and the surrounding capillaries, also causes structural changes in the endothelial cell layer of blood vessels that alter its permeability and its interaction with leukocytes and platelets. These structural changes again cause impaired endothelial cell barrier function resulting in detrimental vascular effects such as adverse vascular permeability with edema, vascular leakage, adverse leukocyte extravasation and hypotension (see also FIG. 1), and may further deteriorate gas exchange from lung to blood and from blood to tissue, and vice versa.

One of the well characterized cytoskeletal changes in response to stress involves the reorganization of the actin cytoskeleton and the formation of stress fibers. Kayali et al., (J Biol Chem (2002) 277(45): 42596-602) describe cytoskeletal changes in pulmonary microvascular endothelial cells in response to hypoxia and potential mechanisms involved in this process. The hypoxia-induced actin redistribution appears to be mediated by components downstream of MAPK p38, which is activated in pulmonary endothelial cells in response to hypoxia. Results indicate that kinase MK2, which is a substrate of p38, becomes activated by hypoxia, leading to the phosphorylation of one of its substrates, HSP27. As another example F-actin rearrangement is also an early event in burn-induced endothelial barrier dysfunction, and HSP27, a target of p38 MAPK/MK2 pathway, plays an important role in actin dynamics. As HSP27 phosphorylation is known to alter actin distribution and thus contractility of cells, Kayali et al., provide that the p38-MK2-HSP27 pathway causes changes in vascular permeability due to actin redistribution, as for example observed in hypoxia.

Taken together these results indicate that tissue damage stimulates the p38-MK2-HSP27 pathway leading to significant alteration in the actin cytoskeleton. It has previously also been shown that inhibition of the p38 MAPK pathway ameliorates vascular dysfunction by significantly reducing endothelial cell contraction (Wang et al, APMIS (2014) 122(9):832).

In recent years also another pathway, the PI3K/AKT/mTOR [phosphatidylinositol 3′ kinase (PI3K), protein kinase B (PKB or AKT) and mammalian target of rapamycin (mTOR)] pathway has been identified to be essential for regulating endothelial cell contractility and Tsuji-Tamura and Ogawa indeed (Journal of Cell Science 2016 129: 1165-1178) identified inhibitors of phosphatidylinositol 3-kinase (PI3K)-Akt-pathway and inhibitors of mammalian target of rapamycin complex 1 (mTORC1) inhibitors as potent inducers of endothelial cell elongation required for restoring vascular permeability governed by vascular endothelial cells. Such elongation is required to fill the gaps that form between endothelial cells when these cells contract after p38-MK2-HSP27 and/or PI3K/AKT/mTOR signaled cytoskeleton reorganization. It is these gaps (again see FIG. 1) through which adverse leakage and adverse extravasation occurs that explains the resulting edema, vascular leakage, adverse leukocyte extravasation and loss of vascular fluid with a risk for hypotension.

Closing of these gaps is in general governed by the ratio of various angiogenic factors such as angiopoietin-2 to angiopoietin-1 at the site of increased vascular permeability, whereby angiopoetin-2 in general induces endothelial cell apoptosis (there with enhancing gap-formation) and angiopoietin-1 counters gap formation by facilitating endothelial cell elongation and gap closure. Inhibition of the p38 pathway, but not of the ERK1/2 pathway, attenuates angiopoetin-2-mediated endothelial cell apoptosis (Li et al, Exp Ther Med. 2018 December; 16(6): 4729-4736. Published online 2018 Oct. 1)). In addition, the PI3K/AKT/mTOR pathway modulates the expression of other angiogenic factors as nitric oxide and angiopoietins (Karar and Mayti, Front. Mol. Neurosci., 2 Dec. 2011, https://doi.org/10.3389/fnmol.2011.00051).

Thus, inhibiting signaling events in the p38/p38-MK2-HSP27 and/or PI3K/AKT/mTOR pathways—that signal cytoskeleton contraction—reduces vascular permeability, and therewith reduces adverse permeability and adverse extravasation, with resulting edema, vascular leakage, adverse leukocyte extravasation and loss of vascular fluid with a risk of hypotension. Formulations, methods and means for obtaining such inhibition are objects of this invention.

The invention provides an aqueous formulation or solution useful in fluid resuscitation and/or addressing issues of vascular permeability of a patient, preferably a human patient, said formulation or solution preferably comprising a source of autophagy inhibiting amino acids, said amino acids for least 50% selected from the group of alanine (in one letter code: A), glutamine (Q), glycine (G), valine (V), leucine (L), isoleucine (I), proline (P) and arginine (R), wherein said source comprises at least one peptide comprising said at least 50% amino acids, wherein said peptide is present as a salt of an organic acid, preferably a salt of maleic acid, more preferably of tartaric acid and most preferably of citric acid. It is preferred that said formulation comprises a least a peptide wherein said amino acids are for at least 75% selected from said group of autophagy inhibiting amino acids. In a preferred embodiment, such a formulation useful in fluid resuscitation and/or addressing issues of vascular permeability of a patient, preferably a human patient, comprises a peptide selected from the group, AQGVLPGQ-maleate (“AQGVLPGQ” disclosed as SEQ ID NO: 1), LQGVLPGQ-maleate (“LQGVLPGQ” disclosed as SEQ ID NO: 2), AQGLQPGQ-maleate (“AQGLQPGQ” disclosed as SEQ ID NO: 3), LQGLQPGQ-maleate (“LQGLQPGQ” disclosed as SEQ ID NO: 4), AQGV-maleate (“AQGV” disclosed as SEQ ID NO: 5), LQGVL-maleate (“LQGVL” disclosed as SEQ ID NO: 6), AQGLQ-maleate (“AQGLQ” disclosed as SEQ ID NO: 7), and LQGLQ-maleate (“LQGLQ” disclosed as SEQ ID NO: 8), preferably selected from the group AQGVLPGQ-acetate (“AQGVLPGQ” disclosed as SEQ ID NO: 1), LQGVLPGQ-acetate (“LQGVLPGQ” disclosed as SEQ ID NO: 2), AQGLQPGQ-acetate (“AQGLQPGQ” disclosed as SEQ ID NO: 3), LQGLQPGQ-acetate (“LQGLQPGQ” disclosed as SEQ ID NO: 4), AQGV-acetate (“AQGV” disclosed as SEQ ID NO: 5), LQGVL-acetate (“LQGVL” disclosed as SEQ ID NO: 6), AQGLQ-acetate (“AQGLQ” disclosed as SEQ ID NO: 7), and LQGLQ-acetate (“LQGLQ” disclosed as SEQ ID NO: 8) more preferably selected from the group AQGVLPGQ-tartrate (“AQGVLPGQ” disclosed as SEQ ID NO: 1), LQGVLPGQ-tartrate (“LQGVLPGQ” disclosed as SEQ ID NO: 2), AQGLQPGQ-tartrate (“AQGLQPGQ” disclosed as SEQ ID NO: 3), LQGLQPGQ-tartrate (“LQGLQPGQ” disclosed as SEQ ID NO: 4), AQGV-tartrate (“AQGV” disclosed as SEQ ID NO: 5), LQGVL-tartrate (“LQGVL” disclosed as SEQ ID NO: 6), AQGLQ-tartrate (“AQGLQ” disclosed as SEQ ID NO: 7), and LQGLQ-tartrate (“LQGLQ” disclosed as SEQ ID NO: 8), more preferably selected from the group AQGVLPGQ-citrate (“AQGVLPGQ” disclosed as SEQ ID NO: 1), LQGVLPGQ-citrate (“LQGVLPGQ” disclosed as SEQ ID NO: 2), AQGLQPGQ-citrate (“AQGLQPGQ” disclosed as SEQ ID NO: 3), LQGLQPGQ-citrate (“LQGLQPGQ” disclosed as SEQ ID NO: 4), AQGV-citrate (“AQGV” disclosed as SEQ ID NO: 5), LQGVL-citrate (“LQGVL” disclosed as SEQ ID NO: 6), AQGLQ-citrate (“AQGLQ” disclosed as SEQ ID NO: 7), and LQGLQ-citrate (“LQGLQ” disclosed as SEQ ID NO: 8). In another preferred embodiment, such a formulation useful in fluid resuscitation and/or addressing issues of vascular permeability of a patient, preferably a human patient, comprises a peptide selected from the group AQGVLPGQ-maleate (“AQGVLPGQ” disclosed as SEQ ID NO: 1), LQGVLPGQ-maleate (“LQGVLPGQ” disclosed as SEQ ID NO: 2), AQGLQPGQ-maleate (“AQGLQPGQ” disclosed as SEQ ID NO: 3), LQGLQPGQ-maleate (“LQGLQPGQ” disclosed as SEQ ID NO: 4), LQGVL-maleate (“LQGVL” disclosed as SEQ ID NO: 6), AQGLQ-maleate (“AQGLQ” disclosed as SEQ ID NO: 7), and LQGLQ-maleate (“LQGLQ” disclosed as SEQ ID NO: 8), preferably selected from the group AQGVLPGQ-acetate (“AQGVLPGQ” disclosed as SEQ ID NO: 1), LQGVLPGQ-acetate (“LQGVLPGQ” disclosed as SEQ ID NO: 2), AQGLQPGQ-acetate (“AQGLQPGQ” disclosed as SEQ ID NO: 3), LQGLQPGQ-acetate (“LQGLQPGQ” disclosed as SEQ ID NO: 4), LQGVL-acetate (“LQGVL” disclosed as SEQ ID NO: 6), AQGLQ-acetate (“AQGLQ” disclosed as SEQ ID NO: 7), and LQGLQ-acetate (“LQGLQ” disclosed as SEQ ID NO: 8), more preferably selected from the group AQGVLPGQ-tartrate (“AQGVLPGQ” disclosed as SEQ ID NO: 1), LQGVLPGQ-tartrate (“LQGVLPGQ” disclosed as SEQ ID NO: 2), AQGLQPGQ-tartrate (“AQGLQPGQ” disclosed as SEQ ID NO: 3), LQGLQPGQ-tartrate (“LQGLQPGQ” disclosed as SEQ ID NO: 4), LQGVL-tartrate (“LQGVL” disclosed as SEQ ID NO: 6), AQGLQ-tartrate (“AQGLQ” disclosed as SEQ ID NO: 7), and LQGLQ-tartrate (“LQGLQ” disclosed as SEQ ID NO: 8), more preferably selected from the group AQGVLPGQ-citrate (“AQGVLPGQ” disclosed as SEQ ID NO: 1), LQGVLPGQ-citrate (“LQGVLPGQ” disclosed as SEQ ID NO: 2), AQGLQPGQ-citrate (“AQGLQPGQ” disclosed as SEQ ID NO: 3), LQGLQPGQ-citrate (“LQGLQPGQ” disclosed as SEQ ID NO: 4), LQGVL-citrate (“LQGVL” disclosed as SEQ ID NO: 6), AQGLQ-citrate (“AQGLQ” disclosed as SEQ ID NO: 7), and LQGLQ-citrate (“LQGLQ” disclosed as SEQ ID NO: 8). In further preferred embodiment, such a formulation useful in fluid resuscitation and/or addressing issues of vascular permeability of a patient, preferably a human patient, comprises a peptide selected from the group AQGVLPGQ-maleate (“AQGVLPGQ” disclosed as SEQ ID NO: 1), LQGVLPGQ-maleate (“LQGVLPGQ” disclosed as SEQ ID NO: 2), AQGLQPGQ-maleate (“AQGLQPGQ” disclosed as SEQ ID NO: 3), and LQGLQPGQ-maleate (“LQGLQPGQ” disclosed as SEQ ID NO: 4), preferably selected from the group AQGVLPGQ-acetate (“AQGVLPGQ” disclosed as SEQ ID NO: 1), LQGVLPGQ-acetate (“LQGVLPGQ” disclosed as SEQ ID NO: 2), AQGLQPGQ-acetate (“AQGLQPGQ” disclosed as SEQ ID NO: 3), and LQGLQPGQ-acetate (“LQGLQPGQ” disclosed as SEQ ID NO: 4), more preferably selected from the group AQGVLPGQ-tartrate (“AQGVLPGQ” disclosed as SEQ ID NO: 1), LQGVLPGQ-tartrate (“LQGVLPGQ” disclosed as SEQ ID NO: 2), AQGLQPGQ-tartrate (“AQGLQPGQ” disclosed as SEQ ID NO: 3), and LQGLQPGQ-tartrate (“LQGLQPGQ” disclosed as SEQ ID NO: 4), more preferably selected from the group AQGVLPGQ-citrate (“AQGVLPGQ” disclosed as SEQ ID NO: 1), LQGVLPGQ-citrate (“LQGVLPGQ” disclosed as SEQ ID NO: 2), AQGLQPGQ-citrate (“AQGLQPGQ” disclosed as SEQ ID NO: 3), and LQGLQPGQ-citrate (“LQGLQPGQ” disclosed as SEQ ID NO: 4). In a further preferred embodiment, such a formulation useful in fluid resuscitation and/or addressing issues of vascular permeability of a patient, preferably a human patient, comprises a peptide selected from the group AQGV-maleate (“AQGV” disclosed as SEQ ID NO: 5), LQGVL-maleate (“LQGVL” disclosed as SEQ ID NO: 6), AQGLQ-maleate (“AQGLQ” disclosed as SEQ ID NO: 7), and LQGLQ-maleate (“LQGLQ” disclosed as SEQ ID NO: 8), preferably selected from the group AQGV-acetate (“AQGV” disclosed as SEQ ID NO: 5), LQGVL-acetate (“LQGVL” disclosed as SEQ ID NO: 6), AQGLQ-acetate (“AQGLQ” disclosed as SEQ ID NO: 7), and LQGLQ-acetate (“LQGLQ” disclosed as SEQ ID NO: 8), more preferably selected from the group AQGV-tartrate (“AQGV” disclosed as SEQ ID NO: 5), LQGVL-tartrate (“LQGVL” disclosed as SEQ ID NO: 6), AQGLQ-tartrate (“AQGLQ” disclosed as SEQ ID NO: 7), and LQGLQ-tartrate (“LQGLQ” disclosed as SEQ ID NO: 8), more preferably selected from the group AQGV-citrate (“AQGV” disclosed as SEQ ID NO: 5), LQGVL-citrate (“LQGVL” disclosed as SEQ ID NO: 6), AQGLQ-citrate (“AQGLQ” disclosed as SEQ ID NO: 7), and LQGLQ-citrate (“LQGLQ” disclosed as SEQ ID NO: 8). In a further preferred embodiment, such a formulation useful in fluid resuscitation and/or addressing issues of vascular permeability of a patient, preferably a human patient, comprises a peptide selected from the group LQGVL-maleate (“LQGVL” disclosed as SEQ ID NO: 6), AQGLQ-maleate (“AQGLQ” disclosed as SEQ ID NO: 7), and LQGLQ-maleate (“LQGLQ” disclosed as SEQ ID NO: 8), preferably selected from the group LQGVL-acetate (“LQGVL” disclosed as SEQ ID NO: 6), AQGLQ-acetate (“AQGLQ” disclosed as SEQ ID NO: 7), and LQGLQ-acetate (“LQGLQ” disclosed as SEQ ID NO: 8), more preferably selected from the group LQGVL-tartrate (“LQGVL” disclosed as SEQ ID NO: 6), AQGLQ-tartrate (“AQGLQ” disclosed as SEQ ID NO: 7), and LQGLQ-tartrate (“LQGLQ” disclosed as SEQ ID NO: 8), more preferably selected from the group LQGVL-citrate (“LQGVL” disclosed as SEQ ID NO: 6), AQGLQ-citrate (“AQGLQ” disclosed as SEQ ID NO: 7), and LQGLQ-citrate (“LQGLQ” disclosed as SEQ ID NO: 8). In another more preferred embodiment, such a formulation useful in fluid resuscitation and/or addressing issues of vascular permeability of a patient, preferably a human patient, comprises an AQGV-maleate (“AQGV” disclosed as SEQ ID NO: 5), preferably an AQGV-acetate (“AQGV” disclosed as SEQ ID NO: 5), more preferably an AQGV-tartrate (“AQGV” disclosed as SEQ ID NO: 5), more preferably an AQGV-citrate (“AQGV” disclosed as SEQ ID NO: 5).

The invention also provides a method for identifying a peptide capable of reducing p38 MAPK kinase activity, such a peptide useful in fluid resuscitation and/or addressing issues of vascular permeability of a patient, preferably a human patient, comprising providing cells, preferably human cells, with a peptide comprising amino acids, said amino acids for at least 50% selected from the group of alanine (in one letter code: A), glutamine (Q), glycine (G), valine (V), leucine (L), isoleucine (I), proline (P) and arginine (R), providing said cells with fMLP and detecting phosphorylation of p38 MAPK in the absence and presence of said peptide at an appropriate time interval, preferably in the order of minutes, most preferably from about half a minute to about 5 minutes e.g. 30 to 600 seconds after provision of fMLP, and comparing the results to determine said peptide's effect on said phosphorylation. Having tested the autophagy inhibiting AQGV-peptide (SEQ ID NO: 5), we detect FPR-activation of FPR-expressing cells with prototype FPR-ligand fMLP to cause rapidly induced and significant (p<0.05; p38 from 60 to 600 sec, PKB at 600 sec) changes in phosphorylation status of PKB (also known as AKT) (FIG. 3a) and p38 MAPK kinases (FIG. 3c), but not (or not detected) in STAT3, JNK (FIG. 3b) and P42/p44MAPK/ERK1,2 (FIG. 3d) kinases. Therewith the invention also provides a method for identifying a peptide capable of reducing PI3K/AKT/mTOR activity, such a peptide useful in fluid resuscitation and/or addressing issues of vascular permeability of a patient, preferably a human patient comprising providing cells with a peptide consisting of amino acids, said amino acids for at least 50% selected from the group of alanine (in one letter code: A), glutamine (Q), glycine (G), valine (V), leucine (L), isoleucine (I), proline (P) and arginine (R), providing said cells with fMLP and detecting phosphorylation of PKB (AKT) in the absence and presence of said peptide at an appropriate time interval, preferably in the order of minutes, most preferably from about half a minute to about 5 minutes, e.g. 30 to 600. AQGV-peptide (SEQ ID NO: 5) effects on p38 MAPK (FIG. 3c) are already detected at 30 seconds after FPR-stimulation, AQGV-peptide (SEQ ID NO: 5) effects on PKB(AKT) follow (FIG. 3a) in a bi-phasic pattern at 300 sec. Both AQGV-peptide (SEQ ID NO: 5) effects on p38 and PKB-mediated signalling last for the full 600 seconds tested whereas the other kinases tested were not affected throughout. As this acute and specific response to treatment shows specific and rapid effects of autophagy-inhibiting-AQGV-peptide (SEQ ID NO: 5) on p38 signaling in the context of regulation of the PI3K/AKT/mTOR pathway, said pathway is governing the balance between proteolysis and proteogenesis regulating cytoskeleton changes affecting vascular permeability. Such activities are not detected in STAT3, JNK (FIG. 3b) and P42/p44MAPK/ERK1,2 (FIG. 3d) kinases tested with AQGV-peptide (SEQ ID NO: 5). It is shown that AQGV-peptide (SEQ ID NO: 5) reduces p38 MAPK kinase activated changes as well as reduces PI3K/AKT/mTOR activated induced changes in cell cytoskeleton reorganization affecting endothelial cell contraction and adverse vascular permeability. The invention also provides a method for identifying a peptide capable of reducing PI3K/AKT/mTOR activity, comprising providing cells with a peptide consisting of amino acids, said amino acids for at least 50% selected from the group of alanine (in one letter code: A), glutamine (Q), glycine (G), valine (V), leucine (L), isoleucine (I), proline (P) and arginine (R), providing said cells with fMLP and detecting phosphorylation of PKB (AKT) in the absence and presence of said peptide at an appropriate time interval, preferably in the order of minutes, most preferably from about half a minute to about 5 minutes, e.g. 30 to 600 seconds after provision of fMLP, and comparing the results to determine said peptide's effect on said phosphorylation. Identified AQGV-peptide (SEQ ID NO: 5) is useful and capable of addressing adverse vascular permeability, such as manifested by edema with vascular leakage, adverse leukocyte extravasation and hypotension in human subjects.

The invention also provides a method for identifying a peptide capable of reducing PI3K/AKT/mTOR activity, comprising providing cells, preferably human cells, with a peptide consisting of amino acids, said amino acids for at least 50% selected from the group of alanine (in one letter code: A), glutamine (Q), glycine (G), valine (V), leucine (L), isoleucine (I), proline (P) and arginine (R), providing said cells with fMLP and detecting phosphorylation of PKB (AKT) in the absence and presence of said peptide at an appropriate time interval, preferably in the order of minutes, most preferably from about half a minute to about 5 minutes, e.g. 30 to 600 seconds after provision of fMLP, and comparing the results to determine said peptide's effect on said phosphorylation. The invention therewith also provides method for identifying a peptide capable of reducing cytoskeleton reorganization, comprising providing cells with a peptide consisting of amino acids, said amino acids for at least 50% selected from the group of alanine (in one letter code: A), glutamine (Q), glycine (G), valine (V), leucine (L), isoleucine (I), proline (P) and arginine (R), and providing said cells with fMLP and detecting phosphorylation of p38 MAPK and/or PKB (AKT) in the absence and presence of said peptide at an appropriate time interval, preferably in the order of minutes, most preferably from about half a minute to about minutes after provision of fMLP, and comparing the results to determine said peptide's effect on said phosphorylation.

The invention relates to a distinct and new class of drugs: autophagy inhibiting compounds that comprise peptides and/or amino acids that target the nutrient sensing system of the mechanistic target of rapamycine, mTOR and inhibit autophagy. Upon testing formyl-peptide related signaling effects of an autophagy inhibiting AQGV-peptide (SEQ ID NO: 5) the peptide was found to unexpectedly attenuate p38/p38-MK2-HSP27 and/or PI3K/AKT/mTOR pathways that govern signal cytoskeleton contraction in modulating vascular permeability. Hence, the current invention relates to the use of an autophagy inhibiting peptide herein also referred to as an AQGV-peptide (SEQ ID NO: 5), and analogues (functional equivalents) thereof, for improving vascular permeability. An autophagy inhibiting peptide or compound herein is defined as a molecule or composition provided with a source of amino acids that comprise at least 50%, more preferably at least 75%, most preferably 100% amino acids selected from the group of autophagy inhibiting amino acids alanine (in the one letter code that is herein used: A), glutamine (Q), glycine (G), valine (V), leucine (L), proline (P), isoleucine (I) and arginine (R). Several of these compounds with autophagy inhibiting peptides composed of (a selection) of above amino acids, are for example disclosed in US2005214943, US2008027007, (see for example herein AQGV (SEQ ID NO: 5), also known as EA-230, LQGV (SEQ ID NO: 9), VLPALP (SEQ ID NO: 10) and related peptides), US2016229890 (see for example RIVPA (SEQ ID NO: 11), also known as IMX942) and related peptides, and US2019315823 (VRLIVAVRIWRR-NH2 (“VRLIVAVRIWRR” disclosed as SEQ ID NO: 12), IDR-1018) and related peptides].

Said amino acids are preferably present in the molecule in the form of a peptide, comprising a string of above amino acids (the peptide), essentially in a linear form (although cyclic or branched forms of peptide are suitable as well). An AQGV-peptide (SEQ ID NO: 5) herein is defined as a autophagy inhibiting peptide comprising at least 50%, more preferably at least 75%, most preferably 100% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, said AQGV-peptide (SEQ ID NO: 5) comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P). It is preferred that an AQGV-peptide (SEQ ID NO: 5) consists of at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO: 5).

Typically, as the invention herein provides a molecular mode-of-action (MoA) of the group of autophagy inhibiting peptides its effects do not necessarily depend on their exact sequence. Instead, their constituent amino acids are meant to provide common household, “no-danger or tissue-repair” signals to the nutrient-sensing system of mTOR; leading to inhibition of autophagy and resulting in resolve of disease. These tissue-repair signal molecules change the balance of proteogenesis versus proteolysis in a cell of and may lead to resolve of disease in three steps:
1 Administered peptide or amino acid fragments thereof are for taken up by amino acid transport, PEPT1/2 transport, by common endocytosis, in the case of vascular cells by elastin receptor mediated endocytosis or phagocytosis.
2 Internalized peptide is hydrolyzed and its amino acids are presented to the nutrient-sensing system of mTOR.
3 Particular amino acids inhibit autophagy, therewith inhibiting proteolysis and leading to proteogenic resolve and pharmaceutical effect.

Various peptides, either derived from breakdown of peptide hormones or assembled as novel synthetic peptide essentially comprising amino acids selected from the group of autophagy inhibiting amino acids, meeting one or more of the characteristics of the above description show, in various animal models in mice or rats to provide potent resolve of excess or adverse—local or systemic-vascular permeability through effects on endothelial cells lining our vasculature. Exploiting the autophagy inhibiting mechanism involved through future clinical application of these autophagy inhibiting compounds and related peptide drugs provides an exciting novel avenue for the rational treatment of disease. However, with several autophagy inhibiting peptide formulations for intravenous application, peptide solubility difficulties have been experienced that decrease availability of autophagy-inhibiting amino acids, necessitating providing stock solutions of peptide in cumbersome large volumes to avoid aggregation of peptide and loss of pharmaceutical effect.

Commonly, pharmaceutical peptide compositions are synthesized using trifluoroacetate as a counter-ion or salt after which trifluoroacetate is exchanged by the counter-ion acetate (from acetic acid) as the pharmaceutical excipient or anion of choice. Acetic acid, also known as ethanoic acid, belongs to the class of organic compounds known as carboxylic acids. Carboxylic acids are compounds containing a carboxylic acid group with the formula —C(═O)OH. Acetic acid exists as a liquid, soluble (in water), and a weakly acidic compound (based on its pKa). Acetic acid has been found in human liver and kidney tissues, and has also been detected in most biological fluids, including feces, urine, breast milk, and saliva. Within the cell, acetic acid is primarily located in the cytoplasm, mitochondria and Golgi. Acetic acid exists in all eukaryotes, ranging from yeast to humans. Acetic acid participates in a number of enzymatic reactions. An acetate is a salt or ester of acetic acid. A salt of acetic acid is known as an acetate. Sodium acetate is generally recognized as safe (GRAS) as a direct human food ingredient. Given all above properties, acetates are generally considered the most suitable option for use in most pharmaceutical peptide-salt formulations and are widely used. With several autophagy inhibiting peptide-acetate formulations for intravenous application, however, peptide solubility difficulties such as undesired aggregation at useful concentrations in aqueous solution have been experienced. Solubility issues and aggregation in the end decrease optional availability of autophagy-inhibiting amino acids, necessitating providing stock solutions of peptide in cumbersome large volumes to avoid aggregation of peptide and loss of pharmaceutical effect.

A great variety of salts and solutes have been screened to determine their influence on aggregation (see for example J. Phys. Chem. B 2013, 117, 27, 8150-8158; J. Am. Chem. Soc. 1972, 94, 4, 1299-1308), revealing that proteins and peptides indeed “salt out” of solution in an anion-specific and concentration-dependent manner. IDR-1018 (VRLIVAVRIWRR-NH2 (“VRLIVAVRIWRR” disclosed as SEQ ID NO: 12)) and related peptides were found to suffer strongly from aggregation, and therewith strongly loose therapeutic effects, in solutions containing large polyatomic anions, such as phosphate, benzoate, nitrate, and citrate. A comparatively small amount of aggregation was observed in solutions containing sulfate and bicarbonate anions as well as chloride and iodide monoatomic anions, and only at ion concentrations above 200 mM. Interestingly, IDR-1018 prepared in sodium acetate did not induce any appreciable aggregation, suggesting that this anion (acetate) may provide an exception to the effect of large polyatomic anions on the aggregation property of synthetic peptides (Cell Chemical Biology, Volume 24, Issue 8 17 Aug. 2017, Pages 969-980.e4).

Aggregation of peptides in solution must be always be considered when designing synthetic peptide therapeutics, as indeed aggregation may hamper biological activities of said peptides and aggregates tend to elicit adverse immune responses. As synthetic peptides, as collections of monomers, are known to aggregate more strongly in solutions containing large polyatomic ions, and such solubility issues are considered not easily undone, it would be useful to identify appropriate salts that help address solubility issues of pharmaceutical preparations with synthetic peptides. Often, the amino acid composition of peptides in question can help predict the solubility of classes of peptide.

Generally, before choosing a solvent and dissolving the peptide, a peptide sequence is studied to determine whether the peptide is acid, basic or neutral and the following steps are generally taken to help predict or design peptide solubility:
Assign a value of −1 to each acidic (charged) residue (D, E, and terminal COOH).
Assign a value of +1 to each basic (charged) residue (K, R and terminal NH₂).
Assign a value of +1 to each H residue at pH<6 and zero at pH>6
Count the total number of charges of the peptide (D, E, K, R, terminal COOH, and NH₂).
Calculate the overall net charge of the peptide.
   Overall net charge <0: Acidic peptide; Try to dissolve it in a basic solution by adding 10% NH₄OH or ammonium bicarbonate in your buffer.
   Overall net charge >0: Basic peptide; Try to dissolve it in an acidic solution by adding 10% acetic acid in your buffer.
   Overall net charge=0: Neutral peptide: If the peptide contains >25% charged residues (e.g., D, K, R, H and E), it is generally soluble in water or aqueous buffers. Below 25% charged residues, it is recommended to use organic solvents (as dimethyl sulfoxide (DMSO), acetonitrile (ACN), dimethylformamide (DMF). Very short peptides consisting of less than five residues are usually soluble in water or aqueous buffer, except when the entire sequence consists of hydrophobic amino acids (e.g., W, L, I, F, M, V, Y). In this case, the use of organic solvents is deemed necessary. Furthermore, neutral peptides containing 50% or more hydrophobic residues (W, L, I, F, M, V, Y, P, A) are generally poorly soluble in aqueous solutions. It is often recommend to dissolve hydrophobic peptides in 100% organic solvent (DMSO, DMF or ACN) and subsequently dilute with water or buffer to the desired concentration. If peptides then aggregate, they need to undergo a freeze-drying step before attempting another solubilisation to reach a lower dilution.

DMSO is the ideal organic solvent for simple biological applications because of its low toxicity, however, in preparing a drug for intravenous (human) use, DMSO, or for that matter ACN or DMF, is undesired. Addition of denaturing agents, such as 6M urea, 6M urea with 20% acetic acid, or 6M guanidine, can help in reducing the aggregation of neutral peptides by disrupting hydrogen bonding network. However, these compounds can interfere with most biological systems and therefore their application is rather limited, and again is undesirable for use in drug development for parenteral use as a whole.

It is a purpose of this disclosure to provide said autophagy-inhibiting amino acids in a most expedient way to a subject deemed in need thereof. Therefore, the invention provides a tartrate or a citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids alanine (A), glutamine (Q), glycine (G), valine (V), leucine (L), isoleucine (I) and proline (P). More preferably, the invention provides a stock solution, preferably aqueous, comprising a peptide-tartrate or a peptide citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids alanine (A), glutamine (Q), glycine (G), valine (V), leucine (L), isoleucine (I) and proline (P).

The invention provides a suitable solution for several autophagy inhibiting peptides to mitigate aggregation of said peptides and identifies tartrate (from tartaric acid, preferably from (+)-tartaric acid) and more preferably citrate (from citric acid) as a suitable counter-ion, pharmaceutical excipient or anion of choice for preparing a salt of an autophagy inhibiting peptide that is a neutral peptide as defined herein above. A variety of salts were screened herein to determine their influence on aggregation of neutral peptide according to the invention, indeed revealing that neutral peptide “salts out” of solution in an anion-specific and concentration-dependent manner. Aggregation points of such salts (the point of concentration below which aggregated peptide-salt tends to resolve), as peptide-sulfate, peptide-maleate, peptide-adenosine monophosphate and peptide-adenosine in aqueous solution were found to show aggravated aggregation in relation to peptide-acetate aggregation, whereas surprisingly tartrate, and more surprisingly peptide-citrate, showed (strongly) reduced aggregation in aqueous solution in comparison to peptide-acetate.

It is preferred that said autophagy inhibiting peptide-salt according to the invention comprises <25% charged residues selected from the group K, H and R. It is more preferred that said autophagy inhibiting peptide comprises <25% charged residues selected from the group D, K, R, H, and E. It is most preferred that said autophagy inhibiting peptide-salt does not comprise residues selected from the group D, K, R, H, and E, avoiding issues of pH incompatibility with fluids for intravenous use. It is furthermore preferred that said solution is an aqueous solution. In a most preferred embodiment, the solution is a so-called stock solution, preferably an aqueous stock solution. A stock solution generally is a concentrated solution of an active substance, herein autophagy inhibiting peptide-salt, that will be diluted to some lower concentration for actual use of said substance, a so-called working solution. Such lower concentration working solutions are for example infusion fluids, e.g. for intravenous or intra-abdominal use to which the peptide is added from the stock solution for administrating therapy to a patient, as often seen in patients at risk of becoming critically ill or already critically ill patients, for example at the intensive care of an hospital or at the battlefield. Under such conditions it is useful, and often considered a requisite, to have the active (peptide) drug available in a small (stock) volume for dilution into the infusion fluid. So-called stock solutions are generally provided and used to save solubilization and preparation time, conserve materials, reduce storage space, and improve the accuracy with which lower concentrated solutions are prepared to work with. Stock solutions of drugs are often prepared and then provided or stored for imminent intravenous use, for example in critically ill patients. However, due to its by default higher peptide concentration, a stock solution with an autophagy inhibiting peptide invariably runs higher risks on peptide drug aggregation than a final working solution. Stock solutions are generally prepared at a concentration well below an aggregation concentration of the salt in question (e.g. 40-50%) to prevent salt-out events under possibly prolonged storage at various ambient conditions. Risk of peptide aggregation (salting-out) is a phenomenon that the invention provides to avoid or mitigate herein with a stock solution according to the invention. Such stock solutions generally are diluted 10- to 100-fold, or more, to provide a suitable working solution. It is however also an object of the present invention to provide working solutions of the peptide-salts according to the invention. Particularly because in the application of the peptides of the invention relatively high amounts/concentrations of the peptide salts typically must be given, it is a prerequisite that the working solutions are far away from salting out points and yet are presented in a relatively small volume.

A variety of salts were screened herein to determine their influence on aggregation of neutral peptide, indeed revealing that neutral peptide “salts out” of solution in an anion-specific and concentration-dependent manner. Peptide-sulfate and peptide-maleate were found to show aggravated aggregation in relation to peptide-acetate aggregation, whereas surprisingly tartrate, and even more surprisingly peptide-citrate, showed (strongly) reduced aggregation in comparison to peptide-acetate.

In a preferred embodiment, the invention provides a (stock)solution of an autophagy inhibiting peptide-acetate or autophagy inhibiting peptide-tartrate or autophagy inhibiting peptide-citrate according to the invention wherein the concentration of said peptide is larger than 0.85 mol/L, more preferably larger than 0.9 mol/L, more preferably larger than 1 mol/L, more preferably larger than 1.2 mol/L, more preferably larger than 1.4 mol/L, more preferably larger than 1.6 mol/L, more preferably larger than 1.8 mol/L. In a more preferred embodiment, the invention provides a stock-solution of said peptide-tartrate or said peptide citrate wherein the concentration of said peptide is in the range of 2 mol/L to 2.5 mol/L. In a more preferred embodiment, the invention provides a stock-solution of said peptide citrate wherein the concentration of said peptide is in the range of 2.5 mol/L to 3 mol/L. In a more preferred embodiment, the invention provides a stock-solution of said peptide citrate wherein the concentration of said peptide is in the range of 3 mol/L to 3.5 mol/L. In a more preferred embodiment, the invention provides a stock-solution of said peptide citrate wherein the concentration of said peptide is in the range of 3.5 mol/L to 4.5 mol/L. In a more preferred embodiment, the invention provides a stock-solution of said peptide citrate wherein the concentration of said peptide is in the range of 4.5 mol/L to 5.5 mol/L. In a more preferred embodiment, the invention provides a stock-solution of said peptide citrate wherein the concentration of said peptide is equal to or larger than 5.5 mol/L. It is preferred that said stock solution is an aqueous solution.

The invention thereby contributes to improved solubility of this distinct and new class of drugs that is emerging: small autophagy inhibiting peptides comprising amino acids that preferentially inhibit autophagy and target the nutrient sensing system of the mechanistic target of rapamycin, mTOR. Typically, peptides are defined as having 50 or less amino acids, for the purpose of this disclosure, proteins are defined as having >50 amino acids. A autophagy inhibiting peptide herein is defined as a linear, branched or circular string of no longer than 50 amino acids that comprises a peptide sequence with at least 50%, more preferably at least 75%, most preferably 100% amino acids selected from the group of autophagy inhibiting amino acids alanine (in one letter code: A), glutamine (Q), glycine (G), valine (V), leucine (L), proline (P), isoleucine (I) and arginine (R).

Molecular mode-of-action (MoA) of this group of peptides does not depend on their exact sequence. Instead, their constituent amino acids provide common household, “no-danger or tissue-repair” signals to the nutrient-sensing system of mTOR; leading to inhibition of autophagy and resulting in resolve of disease.

### AQLPGVI Group (SEQ ID NO: 13)

It is an aspect of this disclosure to provide said autophagy-inhibiting amino acids in a most expedient way to a subject deemed in need thereof. Therefore, the invention provides a peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids alanine (A), glutamine (Q), glycine (G), valine (V), leucine (L), isoleucine (I) and proline (P). More preferably, the invention provides a stock solution, preferably aqueous, comprising a peptide-tartrate or a peptide citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids alanine (A), glutamine (Q), glycine (G), valine (V), leucine (L), isoleucine (I) and proline (P).

Heeding aggregation risk, a vial with a (stock) solution of AQGV-peptide (SEQ ID NO: 5) as defined herein above for use in a clinical trial hitherto contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5), AQGV-peptide-tartrate (“AQGV” disclosed as SEQ ID NO: 5) or AQGV-peptide-citrate (“AQGV” disclosed as SEQ ID NO: 5) now is provided having an amino acid sequence comprising at least 50%, more preferably at least 75%, most preferably at 100% amino acids selected from the group of autophagy inhibiting amino acids alanine (A), glutamine (Q), glycine (G), valine (V), leucine (L), isoleucine (I) and proline (P), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than 5.5 mol/L. It is preferred that said stock solution is an aqueous solution.

### AQLPVG Group (SEQ ID NO: 14)

In another embodiment, the invention provides a peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising 50%, more preferably at least 75%, most preferably at 100% amino acids selected from the group of autophagy inhibiting amino acids alanine (A), glutamine (Q), leucine (L), valine (V) glycine (G) and proline (P). More preferably, the invention provides a stock solution, preferably aqueous, comprising a peptide-tartrate or a peptide citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids alanine (A), glutamine (Q), glycine (G), valine (V), leucine (L), and proline (P). Heeding aggregation risk, a vial with a stock solution of AQGV-peptide (SEQ ID NO: 5) as defined here above for use in a clinical trial hitherto contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5) AQGV-peptide-tartrate (“AQGV” disclosed as SEQ ID NO: 5) or AQGV-peptide-citrate (“AQGV” disclosed as SEQ ID NO: 5) now is provided having an amino acid sequence comprising at least 50%, more preferably at least 75%, most preferably at 100% amino acids selected from the group of autophagy inhibiting amino acids alanine (A), glutamine (Q), glycine (G), valine (V), leucine (L) and proline (P), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than 5.5 mol/L. It is preferred that said stock solution is an aqueous solution.

### AQLP Group (SEQ ID NO: 15)

In another embodiment, the invention provides a peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising 50%, more preferably at least 75%, most preferably at 100% amino acids selected from the group of autophagy inhibiting amino acids alanine (A), glutamine (Q), leucine (L), and proline (P). More preferably, the invention provides a stock solution, preferably aqueous, comprising a peptide-tartrate or a peptide citrate of a preferably recombinant or synthetic, autophagy inhibiting peptide, said peptide having an amino acid sequence comprising 50%, more preferably at least 75%, most preferably at 100% amino acids selected from the group of autophagy inhibiting amino acids alanine (in one letter code: A), glutamine (Q), leucine (L), and proline (P).

Heeding aggregation risk, a vial with a stock solution of AQGV-peptide (SEQ ID NO: 5) as defined here above for use in a clinical trial hitherto contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5), AQGV-peptide-tartrate (“AQGV” disclosed as SEQ ID NO: 5) or AQGV-peptide-citrate (“AQGV” disclosed as SEQ ID NO: now is provided having an amino acid sequence comprising at least 50%, more preferably at least 75%, most preferably at 100% amino acids selected from the group of autophagy inhibiting amino acids alanine (A), glutamine (Q), leucine (L), and proline (P), to contain at least mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than mol/L. It is preferred that said stock solution is an aqueous solution.

### AQGV Group (SEQ ID NO: 5)

In another embodiment the invention provides peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising 50%, more preferably at least 75%, most preferably at 100% amino acids selected from the group of autophagy inhibiting amino acids alanine (A), glutamine (Q), glycine (G) and valine (V). More preferably, the invention provides a stock solution, preferably aqueous, comprising a peptide-tartrate or a peptide citrate of a, preferably recombinant or synthetic, autophagy inhibiting peptide, said peptide having an amino acid sequence comprising 50%, more preferably at least 75%, most preferably at 100% amino acids selected from the group of autophagy inhibiting amino acids alanine (A), glutamine (Q), glycine (G) and valine (V).

Heeding aggregation risk, a vial with a stock solution of AQGV-peptide (SEQ ID NO: 5) as defined here above for use in a clinical trial hitherto typically contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5) AQGV-peptide-tartrate (“AQGV” disclosed as SEQ ID NO: 5) or AQGV-peptide-citrate (“AQGV” disclosed as SEQ ID NO: now is provided having an amino acid sequence comprising at least 50%, more preferably at least 75%, most preferably at 100% amino acids selected from the group of autophagy inhibiting amino acids alanine (A), glutamine (Q), glycine (G) and valine (V), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than mol/L. It is preferred that said stock solution is an aqueous solution.

### LQGV Group (SEQ ID NO: 9)

In yet another embodiment, the invention provides peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids leucine (L), glutamine (Q), glycine (G) and valine (V). More preferably, the invention provides a stock solution, preferably aqueous, comprising a peptide-tartrate or a peptide citrate of a preferably recombinant or synthetic, autophagy inhibiting peptide, said peptide having an amino acid sequence comprising at least 50%, more preferably at least 75%, most preferably at 100% amino acids selected from the group of autophagy inhibiting amino acids leucine, glutamine (Q), glycine (G) and valine (V).

Heeding aggregation risk, a vial with a stock solution of AQGV-peptide (SEQ ID NO: 5) as defined here above for use in a clinical trial hitherto contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-tartrate (“AQGV” disclosed as SEQ ID NO: 5) or AQGV-peptide-citrate (“AQGV” disclosed as SEQ ID NO: 5) now is provided having an amino acid sequence comprising at least 50%, more preferably at least 75%, most preferably at 100% amino acids selected from the group of autophagy inhibiting amino acids leucine (L), glutamine (Q), glycine (G) and valine (V), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than mol/L. It is preferred that said stock solution is an aqueous solution.

### VLPALP Group (SEQ ID NO: 10)

In yet another embodiment, the invention provides peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids leucine (L), alanine (A), proline (P) and valine (V). More preferably, the invention provides a stock solution, preferably aqueous, comprising a peptide-tartrate or a peptide citrate of a, preferably recombinant or synthetic, autophagy inhibiting peptide, said peptide having an amino acid sequence comprising 50%, more preferably at least 75%, most preferably at 100% amino acids selected from the group of autophagy inhibiting amino acids leucine (in one letter code: L), alanine (A), proline (P) and valine (V).

Heeding aggregation risk, a vial with a stock solution of AQGV-peptide (SEQ ID NO: 5) as defined here above for use in a clinical trial hitherto contained no more than (0.8 mol/L) active substrate in solution. According to the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5) AQGV-peptide-tartrate (“AQGV” disclosed as SEQ ID NO: 5) or AQGV-peptide-citrate (“AQGV” disclosed as SEQ ID NO: 5) now is provided having an amino acid sequence comprising at least 50%, more preferably at least 75%, most preferably at 100% amino acids selected from the group of autophagy inhibiting amino acids leucine (L), alanine (A), proline (P) and valine (V), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than 5.5 mol/L. It is preferred that said stock solution is an aqueous solution.

### LIVA Group (SEQ ID NO: 16)

In yet another embodiment, the invention provides peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids leucine (L), isoleucine, alanine (A) and valine (V). More preferably, the invention provides a stock solution, preferably aqueous, comprising a peptide-tartrate or a peptide citrate of a, preferably recombinant or synthetic, autophagy inhibiting peptide, said peptide having an amino acid sequence comprising 50%, more preferably at least 75%, most preferably at 100% amino acids selected from the group of autophagy inhibiting amino acids leucine (L), alanine (A), isoleucine (I) and valine (V).

Heeding aggregation risk, a vial with a stock solution of AQGV-peptide (SEQ ID NO: 5) as defined here above for use in a clinical trial hitherto contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5) AQGV-peptide-tartrate (“AQGV” disclosed as SEQ ID NO: 5) or AQGV-peptide-citrate (“AQGV” disclosed as SEQ ID NO: 5) now is provided having an amino acid sequence comprising at least 50%, more preferably at least 75%, most preferably at 100% amino acids selected from the group of autophagy inhibiting amino acids leucine (L), isoleucine, alanine (A) and valine (V), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than mol/L. It is preferred that said stock solution is an aqueous solution.

### PIVA Group (SEQ ID NO: 17)

In yet another embodiment, the invention provides peptide, preferably a salt of an organic acid, such as a maleate, more preferably an acetate, more preferably a tartrate, most preferably a citrate of a, preferably recombinant or synthetic, autophagy inhibiting peptide, said peptide having an amino acid sequence comprising at least 50%, more preferably at least 75%, most preferably at 100% amino acids selected from the group of autophagy inhibiting amino acids proline (P), isoleucine (I), alanine (A) and valine (V). More preferably, the invention provides a stock solution, preferably aqueous, comprising a peptide-tartrate or a peptide citrate of a, preferably recombinant or synthetic, autophagy inhibiting peptide, said peptide having an amino acid sequence comprising 50%, more preferably at least 75%, most preferably at 100% amino acids selected from the group of autophagy inhibiting amino acids proline (P), isoleucine (I), alanine (A) and valine (V).

Heeding aggregation risk, a vial with a stock solution of AQGV-peptide (SEQ ID NO: 5) as defined here above for use in a clinical trial hitherto typically contained no more than (0.8 mol/L) active substrate in solution. According to the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5) AQGV-peptide-tartrate (“AQGV” disclosed as SEQ ID NO: 5) or AQGV-peptide-citrate (“AQGV” disclosed as SEQ ID NO: 5) now is provided having an amino acid sequence comprising at least 50%, more preferably at least 75%, most preferably at 100% amino acids selected from the group of autophagy inhibiting amino acids proline (P), isoleucine (I), alanine (A) and valine (V), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than mol/L. It is preferred that said stock solution is an aqueous solution.

In a preferred embodiment, it is preferred that said stock solution is an aqueous solution of autophagy inhibiting amino acids comprising a dipeptide AQ, QQ, LQ, GQ, PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QLG, QGG, QPG, QVG, LAG, LGG, LPG, LVG or a tetrapeptide AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture of at least any two thereof.

In another preferred embodiment, the invention provides peptide, preferably a salt of an organic acid, such as a maleate, more preferably an acetate, more preferably a tartrate, most preferably a citrate of a, preferably recombinant or synthetic, autophagy inhibiting peptide, said peptide comprising at least 50% of dipeptide AQ QQ, LQ, GQ PQ VQ AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide AQGV (SEQ ID NO: 5), AQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture thereof, more preferably at least 75%, most preferably 100% of dipeptide AQ, QQ, LQ, GQ PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QLG, QGG, QPG, QVG, LAG, LGG, LPG, LVG or a tetrapeptide AQGV (SEQ ID NO: 5), QAGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture thereof. More preferably, the invention provides a stock solution, preferably aqueous, comprising a peptide-tartrate or a peptide citrate of a, preferably recombinant or synthetic, autophagy inhibiting peptide, said peptide comprising dipeptide AQ, QQ, LQ GQ PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QLG, QGG, QPG, QVG, LAG, LGG, LPG, LVG or a tetrapeptide AQGV (SEQ ID NO: 5), QAGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture thereof.

Heeding aggregation risk, a vial with a stock solution of AQGV-peptide (SEQ ID NO: 5) comprising dipeptide AQ QQ, LQ GQ PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QLG, QGG, QPG, QVG, LAG, LGG, LPG, LVG or a tetrapeptide AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture of at least any two thereof as defined here above for use in a clinical trial hitherto typically contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5) AQGV-peptide-tartrate (“AQGV” disclosed as SEQ ID NO: 5) or AQGV-peptide-citrate (“AQGV” disclosed as SEQ ID NO: 5) now is provided comprising dipeptide AQ, QQ, LQ GQ PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QLG, QGG, QPG, QVG, LAG, LGG, LPG, LVG or a tetrapeptide AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture of two or more thereof to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5).

In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than 5.5 mol/L. It is preferred that said stock solution is an aqueous solution.

It is preferred that a peptide according to the invention has a peptide sequence with length of 2-40 amino acids, preferably 3-30 amino acids, preferably 4-20 amino acids. It is most preferred that said peptide according to the invention has a peptide sequence that comprises at least 6 amino acids, in particular when at least 4 of those inhibit autophagy. A maximum length of a peptide-tartrate or peptide citrate according to the invention preferably comprises at most 50 amino acids, more preferably at most 40 amino acids, more preferably at most 30 amino acids, more preferably at most 20 amino acids, more preferably at most 15 amino acids, more preferably at most 12 amino acids, most preferably at most 9 amino acids.

The invention also provides an aqueous solution useful in fluid resuscitation and/or addressing issues of vascular permeability of a patient that is prepared with a stock solution according to the invention. It is preferred that such an aqueous solution addressing issues of vascular permeability is provided with an autophagy inhibiting peptide according to the invention has a peptide sequence with length of 2-40 amino acids, preferably 3-30 amino acids, preferably 4-amino acids. It is most preferred that said aqueous solution according to the invention is provided with a peptide that comprises at least 6 amino acids, in particular when at least 4 of those inhibit autophagy. It is more preferred that said aqueous solution according to the invention is provided with a peptide-tartrate or peptide citrate according to the invention that preferably comprises at most 50 amino acids, more preferably at most 40 amino acids, more preferably at most 30 amino acids, more preferably at most 20 amino acids, more preferably at most 15 amino acids, more preferably at most 12 amino acids, most preferably at most 9 amino acids. Such an aqueous solution according to the invention is particularly useful wherein said patient is a human, preferably wherein said patient is considered critically ill, and typically useful in treatment of a disease associated with increased vascular permeability, in particular for delaying vasopressor use or avoiding such use in treatment of increased vascular permeability. Especially when used in prevention or treatment of fluid overload, or as resuscitation fluid, an aqueous solution with an autophagy inhibiting peptide according to the invention that is considered a crystalloid is preferred, providing for a liberal fluids approach consisting of a larger volume of initial fluid, if preferred over the first 6 hours and even later use of vasopressors, if needed at all. Alternatively, use of an aqueous solution, optionally a colloid solution, with an autophagy inhibiting peptide provided herein above and allowing a restrictive fluids approach may postpone or limit said resuscitation therapy's reliance on vasopressor infusions.

The invention also provides a method for reducing p38 MAPK kinase activity leading to cytoskeleton reorganization, comprising providing cells, preferably having a formyl-peptide-receptor associated with their surface, with a source of autophagy inhibiting amino acids, preferably wherein said source is an AQGV-peptide (SEQ ID NO: 5) as provided herein, said amino acids for least 50% selected from the group of alanine (in one letter code: A), glutamine (Q), glycine (G), valine (V), leucine (L), isoleucine (I), proline (P) and arginine (R).

The invention provides a method for reducing formyl-peptide-receptor (FPR) mediated p38 MAPK kinase activity, comprising providing cells, preferably having a formyl-peptide-receptor associated with their surface, with a source of autophagy inhibiting amino acids, preferably wherein said source is an AQGV-peptide (SEQ ID NO: 5) as provided herein, said amino acids for least 50% selected from the group of alanine (in one letter code: A), glutamine (Q), glycine (G), valine (V), leucine (L), isoleucine (I), proline (P) and arginine (R).

The invention provides a method for reducing PI3K/AKT/mTOR activity leading to cytoskeleton reorganization, comprising providing cells, preferably having a formyl-peptide-receptor associated with their surface, with a source of autophagy inhibiting amino acids, preferably wherein said source is an AQGV-peptide (SEQ ID NO: 5) as provided herein, said amino acids for least 50% selected from the group of alanine (in one letter code: A), glutamine (Q), glycine (G), valine (V), leucine (L), isoleucine (I), proline (P) and arginine (R).

The invention provides a method for reducing formyl-peptide-receptor (FPR) mediated PI3K/AKT/mTOR activity, comprising providing cells, preferably having a formyl-peptide-receptor associated with their surface, with a source of autophagy inhibiting amino acids, preferably wherein said source is an AQGV-peptide (SEQ ID NO: 5) as provided herein above and below, said amino acids for least 50% selected from the group of alanine (in one letter code: A), glutamine (Q), glycine (G), valine (V), leucine (L), isoleucine (I), proline (P) and arginine (R).

The invention provides a method for reducing cytoskeleton reorganization, comprising providing cells, preferably having a formyl-peptide-receptor associated with their surface, with a source of autophagy inhibiting amino acids, preferably wherein said source is an AQGV-peptide (SEQ ID NO: 5) as provided herein, said amino acids for least 50% selected from the group of alanine (in one letter code: A), glutamine (Q), glycine (G), valine (V), leucine (L), isoleucine (I), proline (P) and arginine (R).

The invention provides a method for reducing formyl-peptide-receptor (FPR) mediated cytoskeleton reorganization, comprising providing cells, preferably having a formyl-peptide-receptor associated with their surface, with a source of autophagy inhibiting amino acids, preferably wherein said source is an AQGV-peptide (SEQ ID NO: 5) as provided herein, said amino acids for least 50% selected from the group of alanine (in one letter code: A), glutamine (Q), glycine (G), valine (V), leucine (L), isoleucine (I), proline (P) and arginine (R).

The invention provides a method for modifying vascular permeability comprising providing cells, preferably having a formyl-peptide-receptor associated with their surface, with a source of autophagy inhibiting amino acids, preferably wherein said source is an AQGV-peptide (SEQ ID NO: 5) as provided herein, selected from the group of alanine (in one letter code: A), glutamine (Q), glycine (G), valine (V), leucine (L), isoleucine (I), proline (P) and arginine (R).

The invention provides a method for improving tissue repair comprising providing cells, preferably having a formyl-peptide-receptor associated with their surface, with a source of autophagy inhibiting amino acids, preferably wherein said source is an AQGV-peptide (SEQ ID NO: 5) as provided herein, selected from the group of alanine (in one letter code: A), glutamine (Q), glycine (G), valine (V), leucine (L), isoleucine (I), proline (P) and arginine (R).

The invention provides a method according to the invention, wherein said peptide comprising said autophagy inhibiting amino acids comprises a dipeptide AQ, QQ, LQ GQ, PQ VQ AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV, (SEQ ID NO: 35) or a mixture of at least two thereof.

The invention provides a method according to the invention wherein said source of autophagy inhibiting amino acids, preferably wherein said source is an AQGV-peptide (SEQ ID NO: 5) as provided herein, is a peptide comprising a dipeptide selected from the group AQ QQ, LQ GQ PQ VQ AL, LL, QL, GL, PL, VL, QA, QL, QG, QP, QV, LA, LG, LP, LV, a tripeptide selected from the group AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide selected from the group AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV, (SEQ ID NO: 35) or a mixture thereof connected to said antibody-like molecule through a peptide bond.

The invention provides a method according to the invention, wherein said AQGV-peptide (SEQ ID NO: 5) comprises a dipeptide selected from the group AQ QQ, LQ GQ PQ, VQ, AL, LL, QL, GL, PL, VL, QA, QL, QG, QP, QV, LA, LG, LP, LV, a tripeptide selected from the group AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide selected from the group selected from the group AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35) or a mixture thereof.

The invention provides an AQGV-peptide (SEQ ID NO: 5) for use in reducing, preferably formyl-peptide-receptor (FPR) mediated, p38 MAPK kinase and/or PKB activity and/or cytoskeleton reorganization activity, said molecule comprising a source of autophagy inhibiting amino acids, preferably wherein said source is an AQGV-peptide (SEQ ID NO: 5) as provided herein, selected from the group of alanine (in one letter code: A), glutamine (Q), glycine (G), valine (V), leucine (L), isoleucine (I), proline (P) and arginine.

The invention provides an AQGV-peptide (SEQ ID NO: 5) for use in improving tissue repair, said molecule comprising a source of autophagy inhibiting amino acids, preferably wherein said source is an AQGV-peptide (SEQ ID NO: 5) as provided herein, selected from the group of alanine (in one letter code: A), glutamine (Q), glycine (G), valine (V), leucine (L), isoleucine (I), proline (P) and arginine.

The invention provides an AQGV-peptide (SEQ ID NO: 5) for use in modifying vascular permeability, said molecule comprising a source of autophagy inhibiting amino acids, preferably wherein said source is an AQGV-peptide (SEQ ID NO: 5) as provided herein, selected from the group of alanine (in one letter code: A), glutamine (Q), glycine (G), valine (V), leucine (L), isoleucine (I), proline (P) and arginine.

The invention provides an AQGV-peptide (SEQ ID NO: 5) for use according to the invention, wherein said source of autophagy inhibiting amino acids, preferably wherein said source is an AQGV-peptide (SEQ ID NO: 5) as provided herein, is a peptide comprising a dipeptide selected from the group AQ, QQ, LQ, GQ, PQ, VQ, AL, LL, QL, GL, PL, VL, QA, QL, QG, QP, QV, LA, LG, LP, LV, a tripeptide selected from the group AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide selected from the group AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35) or a mixture thereof, for use in reducing, preferably formyl-peptide-receptor (FPR) mediated, p38 MAPK kinase and/or PKB activity and/or cytoskeleton reorganization activity.

A peptide comprising xGxxPG and a peptide selected from a dipeptide selected from the group AQ, QQ, LQ, GQ, PQ, VQ, AL, LL, QL, GL, PL, VL, QA, QL, QG, QP, QV, LA, LG, LP, LV, a tripeptide selected from the group AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide selected from the group AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35) or a mixture thereof, for use in reducing, preferably formyl-peptide-receptor (FPR) mediated, p38 MAPK kinase and/or PKB activity and/or cytoskeleton reorganization activity.

The invention provides an AQGV-peptide (SEQ ID NO: 5) comprising at least 7 amino acids and at most 30 amino acids comprising a sequence of the formula φn xGxxPG, or xGxxPG φn, or φn xGxxPG φm wherein x is a naturally occurring amino acid, φ is an autophagy inhibiting amino acid and n=an integer from 1 to 24 and m is an integer from 1-23, whereby n+m is no greater than 24, for use in reducing, preferably formyl-peptide-receptor (FPR) mediated, p38 MAPK kinase and/or PKB activity and/or cytoskeleton reorganization activity.

The invention provides an AQGV-peptide (SEQ ID NO: 5) according to the invention wherein φn and/or φm comprise AQ, QQ, LQ, GQ, PQ, VQ, AL, LL, QL, GL, PL, VL, QA, QL, QG, QP, QV, LA, LG, LP, LV, AQG, QQG, LQG, GQG, PQG, VQG, ALG, LLG, QLG, GLG, PLG, VLG, QAG, Q0LG, QGG, QPG, QVG, LAG, LGG, LPG, LVG, AQGV (SEQ ID NO: 5), QQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35) or various mixtures thereof.

The invention further provides a pharmaceutical formulation comprising an AQGV-peptide (SEQ ID NO: 5) and a peptide selected from a dipeptide selected from the group AQ, QQ, LQ GQ PQ, VQ, AL, LL, QL, GL, PL, VL, QA, QL, QG, QP, QV, LA, LG, LP, LV, and/or a tripeptide selected from the group AQG, QQG, LQG, GQG, PQG, VQG, ALG, LLG, QLG, GLG, PLG, VLG, QAG, Q0LG, QGG, QPG, QVG, LAG, LGG, LPG, LVG and/or a tetrapeptide selected from the group AQGV (SEQ ID NO: 5), QQGV (SEQ ID NO: 18), LQGV (SEQ ID NO: 9), GQGV (SEQ ID NO: 19), PQGV (SEQ ID NO: VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture thereof, for use in reducing, preferably formyl-peptide-receptor (FPR) mediated, p38 MAPK kinase and/or PKB activity and/or cytoskeleton reorganization activity, and at least one pharmaceutically acceptable excipient.

The invention further provides a pharmaceutical formulation comprising a peptide according to the invention and at least one pharmaceutically acceptable excipient.

The invention also provides a method for producing a AQGV-peptide (SEQ ID NO: 5) according to the invention comprising synthesizing said peptide with an automated peptide synthesizer

In a much preferred embodiment, the invention provides a peptide-tartrate or peptide-citrate according to the invention that comprises a tartrate or citrate of AQGVLPG (SEQ ID NO: 36), AQGVLP (SEQ ID NO: 37), AQLP (SEQ ID NO: 15), AQGV (SEQ ID NO: 5) or LQGV (SEQ ID NO: 9). In a most preferred embodiment the invention provides a tartrate of a tetrapeptide, wherein the tetrapeptide is AQGV (SEQ ID NO: 5) or LQGV (SEQ ID NO: 9). Said tartrate or citrate of a peptide according to the invention may be a tartrate or citrate salt or ester of the peptide, tartrate or citrate salt is preferred. The invention also provides a composition comprising a peptide or peptide salt according to the invention together with a pharmaceutically acceptable excipient. A preferred excipient for intravenous use is 0.9% NaCl.

The invention also provides an aqueous solution comprising a source of autophagy inhibiting amino acids, said amino acids for least 50% selected from the group of alanine (in one letter code: A), glutamine (Q), glycine (G), valine (V), leucine (L), isoleucine (I), proline (P) and arginine (R), wherein said source comprises at least one peptide comprising said at least 50% amino acids, wherein said peptide is present as a salt of an organic acid, preferably a salt of maleic acid, more preferably of tartaric acid and most preferably of citric acid.

### FIGURE LEGENDS

FIG. 1 Formyl-peptide-receptor mediated vascular permeability after cell and tissue trauma.

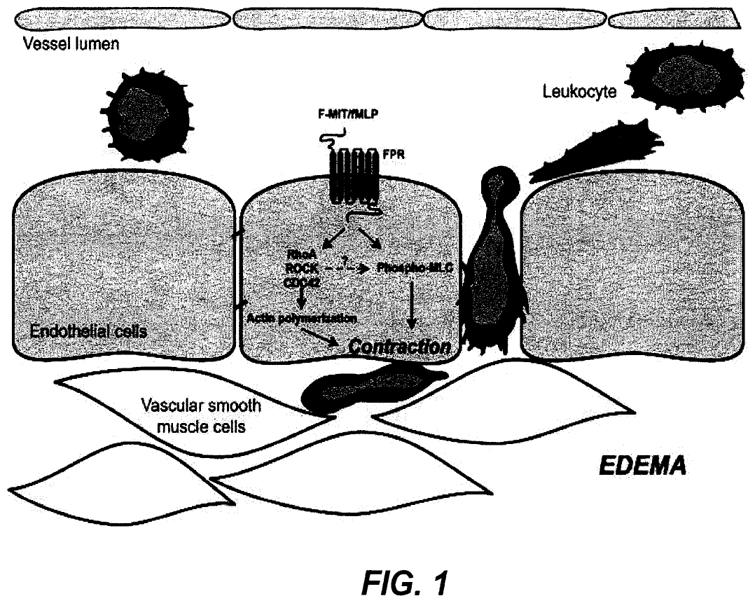

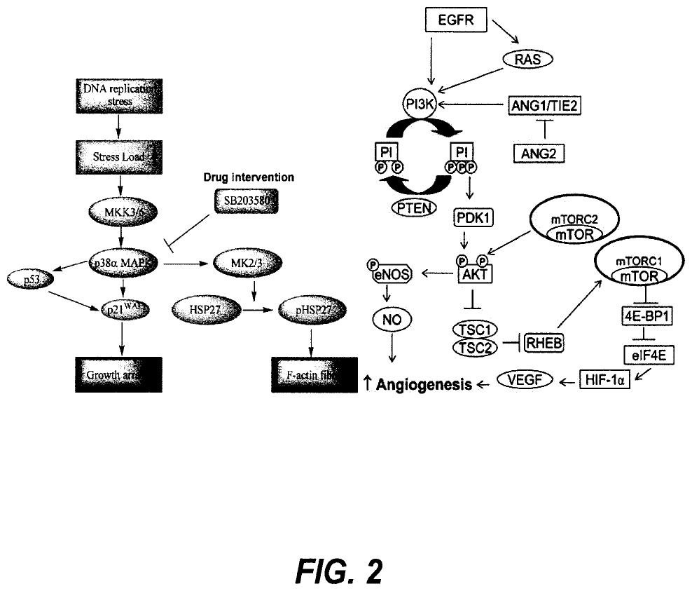

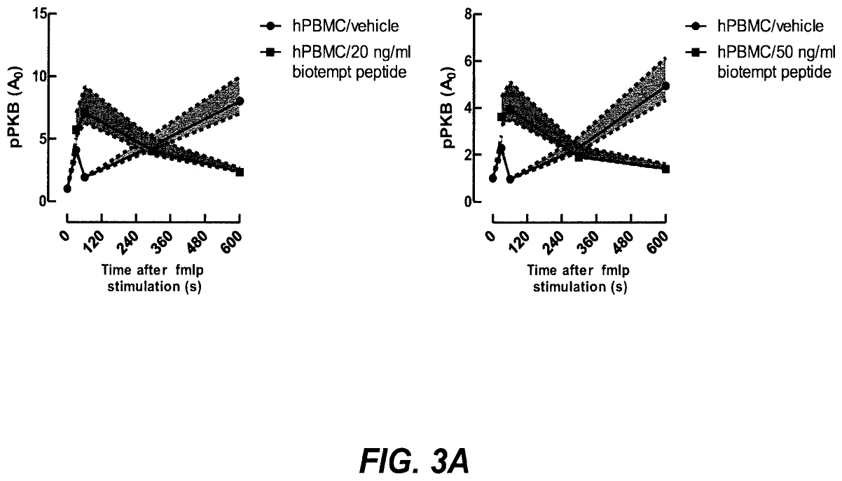

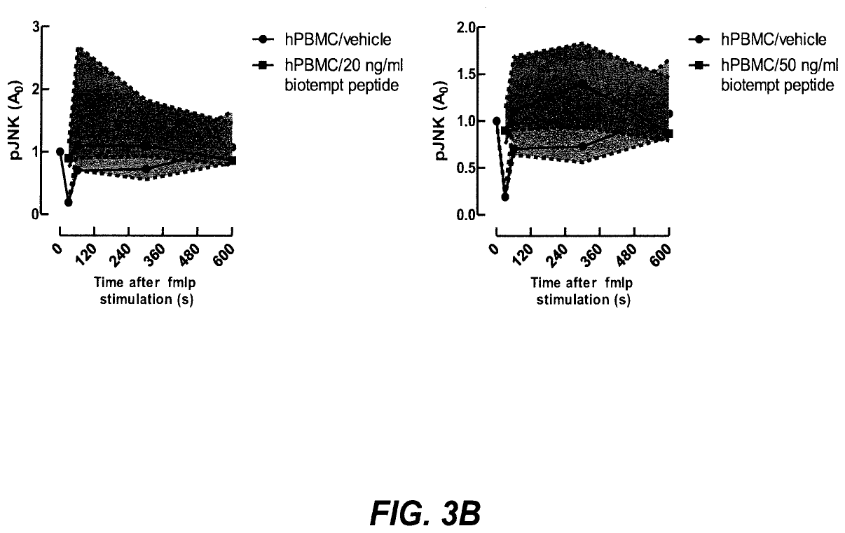

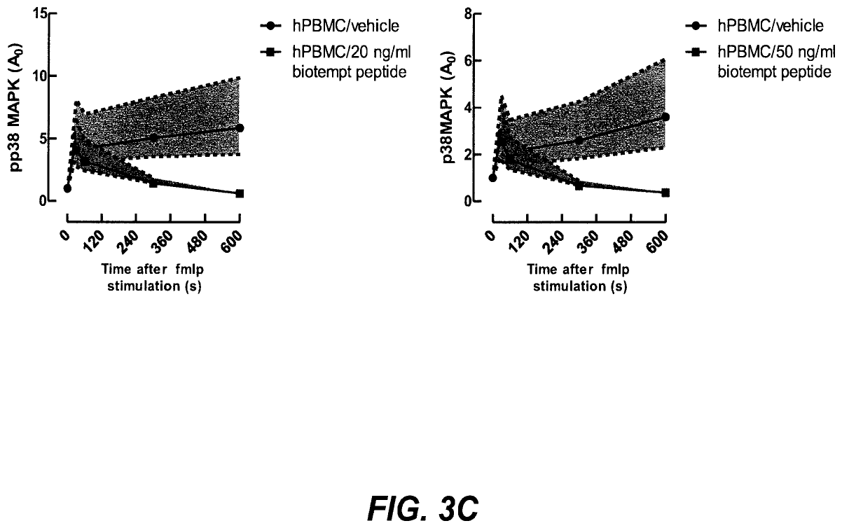

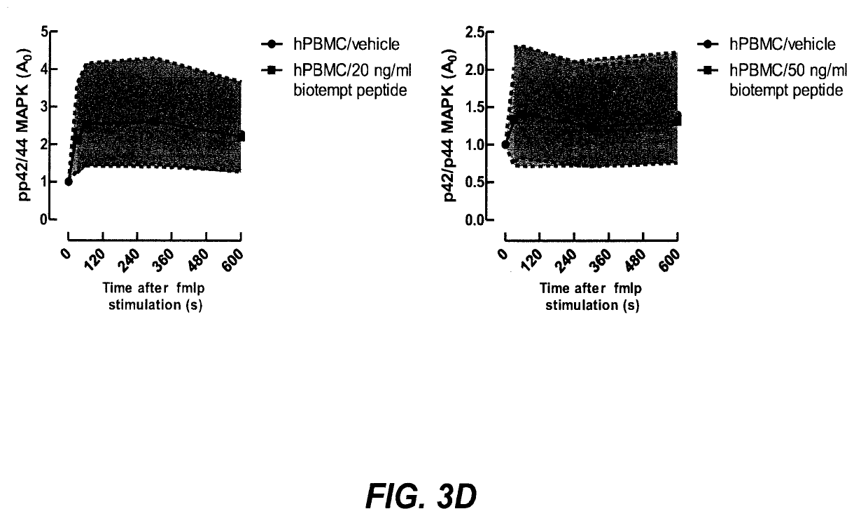

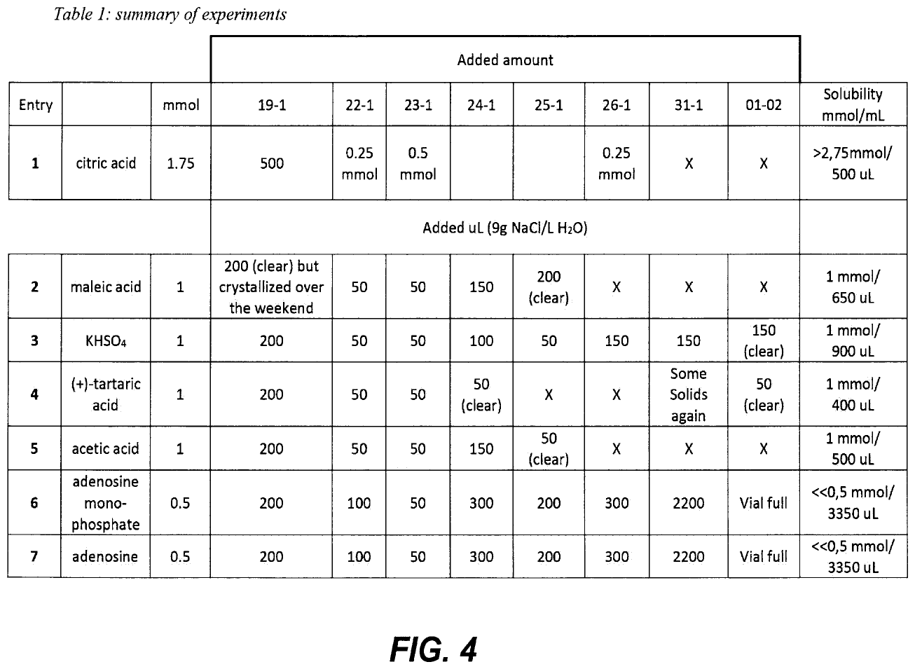

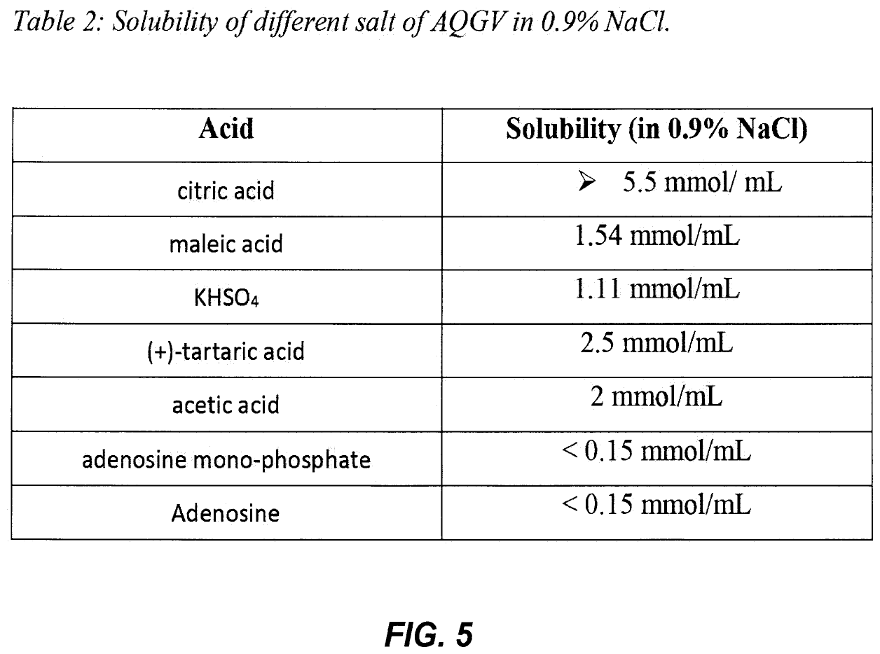

Mitochondrial N-formyl peptides (F-MIT) released from trauma/cell damage activate formyl peptide receptor (FPR) leading to changes in endothelial cell cytoskeleton which subsequently induces endothelial contraction and vascular permeability, leukocyte extravasation and hypotension. N-Formyl peptides are common molecular signatures of bacteria and mitochondria that activate the formyl peptide receptor (FPR). FPR activation by mitochondrial N-formyl peptides (F-MIT) elicits changes in cytoskeleton-regulating proteins in endothelial cells that lead to increased endothelial cell contractility with increased vascular leakage and extravasation of leukocytes. FPR activation via mitochondrial N-formyl peptides (F-MIT) originating from tissue damage after injury such as trauma is a key contributor to impaired barrier function following cell and tissue injury or trauma, resulting in detrimental vascular effects such as adverse vascular permeability with edema, vascular leakage, adverse leukocyte extravasation and hypotension.

FIG. 2

Graphic description of p38-MK2-HSP27 pathway (left) and P113K/AKT/mTOR pathway (right) involved in regulation of endothelial cell-cytoskeleton organization.

FIG. 3

Formyl-peptide-receptor mediated peptide effects.

FPR-activation of FPR-expressing cells with prototype FPR-ligand fMLP causes rapidly induced and significant (p<0.05; p38 from 60 to 600 sec, PKB at 600 sec) changes in phosphorylation status of PKB (also known as AKT) (FIG. 3a) and p38 MAPK kinases (FIG. 3c), but not (or not detected) in STAT3, JNK (FIG. 3b) and P42/p44MAPK/ERK1,2 (FIG. 3d) kinases. AQGV-peptide (SEQ ID NO: 5) effects on p38 MAPK (FIG. 3c) are already detected at 30 seconds after FPR-stimulation, AQGV-peptide (SEQ ID NO: 5) effects on PKB(AKT) follow (FIG. 3a) in a bi-phasic pattern at 300 sec. Both AQGV-peptide (SEQ ID NO: 5) effects on p38 and PKB-mediated signalling last for the full 600 seconds tested whereas the other kinases tested were not affected throughout. This acute and specific response to treatment shows specific and rapid effects of autophagy-inhibiting-AQGV-peptide (SEQ ID NO: 5) on p38 signaling in the context of regulation of the PI3K/AKT/mTOR pathway. Said pathway is governing the balance between proteolysis and proteogenesis regulating cytoskeleton changes affecting vascular permeability. It is shown that AQGV-peptide (SEQ ID NO: 5) reduces p38 MAPK kinase activated changes as well as reduces PI3K/AKT/mTOR activated induced changes in cell cytoskeleton reorganization affecting endothelial cell contraction and adverse vascular permeability. AQGV-peptide (SEQ ID NO: 5) is useful and capable of addressing adverse vascular permeability, such as manifested by edema with vascular leakage, adverse leukocyte extravasation and hypotension in human subjects.

FIG. 4

Overview of solubility experiments with results.

FIG. 5

Based on the results depicted in FIG. 4 the concentration below which an aggregated peptide-salt tends to resolve of the neutral-peptides salts screened were determined (aggregation points). It can be concluded that changing the anion significantly influences the solubility characteristics of AQGV (SEQ ID NO: 5). Higher solubility (solubility in 0.9% NaCl) and therewith higher aggregation points were observed for the AQGV-citric acid (“AQGV” disclosed as SEQ ID NO: 5) (AQGV-citrate (“AQGV” disclosed as SEQ ID NO: 5)) and -tartaric acid (AQGV-tartrate (“AQGV” disclosed as SEQ ID NO: 5)) salt, whereas maleic acid and KHSO₄ salts showed lower solubility, compared to AQGV-Ac (“AQGV” disclosed as SEQ ID NO: 5). Using adenosine-monophosphate or adenosine in our hands did not provide solubility. Citric acid seems to be a special case. Highly concentrated solution does not crystallize or aggregate but tend to form a highly viscous solution.

### DETAILED DESCRIPTION

### Autophagy Inhibiting Peptides

### One Letter Code

In describing protein or peptide composition, structure and function herein, reference is made to amino acids. In the present specification, amino acid residues are identified by using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; Ile: isoleucine residue; Lys: lysine residue; Leu: leucine residue; Met: methionine residue; Asn: asparagine residue; Pro: proline residue; Gln: glutamine residue; Arg: arginine residue; Ser: serine residue; Thr: threonine residue; Val: valine residue; Trp: tryptophane residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

### Peptides

Peptide shall mean herein a natural biological or artificially manufactured (synthetic) short chain of amino acid monomers linked by peptide (amide) bonds. Glutamine peptide shall mean herein a natural biological or artificially manufactured (synthetic) short chain of amino acid monomers linked by peptide (amide) bonds wherein one of said amino acid monomers is a glutamine. Chemically synthesized peptides generally have free N- and C-termini. N-terminal acetylation and C-terminal amidation reduce the overall charge of a peptide; therefore, its overall solubility might decrease. However, the stability of the peptide could also be increased because the terminal acetylation/amidation generates a closer mimic of the native protein. These modifications might increase the biological activity of a peptide and are herein also provided.

### Peptide Synthesis

In this application, peptides are either synthesized by classically known chemical synthesis on a solid support (Ansynth BV, Roosendaal, The Netherlands) or in solution (Syncom BV, Groningen, The Netherlands and Diosynth BV, Oss, The Netherlands). Pharmaceutical peptide compositions may be synthesized using trifluoroacetate as a counter-ion or salt after which trifluoroacetate is exchanged by a counter-ion such as maleate (from maleic acid), acetate (from acetic acid), tartrate (from tartaric acid) or citrate (from citric acid). The drug substance of AQGV (SEQ ID NO: 5) (EA-230) for use in pre-clinical and clinical human studies has been manufactured by Organon N.V (formerly Diosynth B.V.), (Oss, The Netherlands), whereas filling and finishing of the final product has been performed by Octoplus Development, Leiden (The Netherlands). Molecular weight of EA-230 (AQGV (SEQ ID NO: 5)) is 373 g/mol).

### Determination of Chemotactic Activity.

Human U937 monocytic cells are purchased from the American Type Culture Collection (ATCC catalog number CRL-1593.2, Manassas, Va). Cells are maintained in suspension culture in T-75 flasks containing RPMI 1640 medium supplemented with 10% fetal calf serum and antibiotics, and cultures are split every 3 to 5 days. Three days before use in chemotaxis assays, U937 cells are stimulated to differentiate along the macrophage lineage by exposure to 1 mmol/L dibutyryl cyclic adenosine monophosphate (dbcAMP; Sigma Chemical Co), as described. Cells are washed three times to remove culture medium and then resuspended in chemotaxis medium (Dulbecco's modified essential medium supplemented with 1% lactalbumin hydrolysate) for plating into assay chambers at a final concentration of 2.5×106 cells/mL. Chemotaxis assays are performed in 48-well microchemotaxis chambers (Neuro Probe, Cabin John, Md). The bottom wells of the chamber are filled with 25 mL of the chemotactic stimulus (or medium alone) in triplicate. An uncoated 10-mm-thick polyvinylpyrrolidone-free polycarbonate filter with a pore size of 5 mm is placed over the samples (Neuro Probe). The silicon gasket and the upper pieces of the chamber are applied, and 50 mL of the monocyte cell suspension are placed into the upper wells. Chambers are incubated in a humidified 5% CO2 atmosphere for 3 hours at 37° C., and nonmigrated cells are gently wiped away from the upper surface of the filter. The filter is immersed for 30 seconds in a methanol-based fixative and stained with a modified Wright-Giemsa technique (Protocol Hema 3 stain set; Biochemical Sciences, Inc, Swedesboro, NJ) and then mounted on a glass slide. Cells that are completely migrated through the filter are counted under light microscopy, with 3 random high-power fields (HPF; original magnification ×400) counted per well. Human monocytes are isolated from freshly drawn blood of healthy volunteers using serial Ficoll/Pelastin receptor complex (ERC)oll gradient centrifugation, as described elsewhere. Cells are cultured for 16 hours in RPMI-1640 media supplemented with 0.5% human serum to become quiescent after isolation. Purity of the cells is >95% as determined by flow cytometry analysis. Monocyte chemotaxis is assayed in a 48-well microchemotaxis chamber (Neuroprobe, Gaithersburg, MD) in serum-free media. Wells in the upper and lower chamber are separated by a polyvinylpyrrolidone-free polycarbonate membrane (pore size 5 μm; Costar). Freshly isolated monocytes at a density of 5×105/mL are incubated for 2.5 hours with recombinant C-peptide (Sigma), before migrated cells on the bottom face of the filter are stained and counted under the light microscope. Maximal chemotactic activity is measured with 0.1 mmol/L N-formyl-methionyl-leucyl-phenylalanine (f-MLF; Sigma Chemical Co), and checkerboard analysis is used to distinguish chemotaxis from chemokinesis.

### Cell Isolation

Blood is drawn from healthy volunteers into tubes containing citrate as an anticoagulant. Neutrophils are isolated by using a Polymorphprep kit (Nicomed, Oslo, Norway) according to the manufacturer's instructions; monocytes are purified with magnetic beads (Miltenyi Biotech). The purity of the cells, as assessed by flow cytometry (anti-CD45, 14, DR, and CD66b), is >93%. For each cell type, samples from two different donors are examined.

Distribution and elimination of intravenously injected [14C]-AQGV (“AQGV” disclosed as SEQ ID NO: 5) from mice

The present study, which was conducted by TNO Biosciences, Utrechtseweg, Netherlands, was designed to provide data on the distribution and metabolism of [14C]-AQGV (“AQGV” disclosed as SEQ ID NO: 5) (Ala-Gln-[1-¹⁴C]Gly-Val (“AQGV” disclosed as SEQ ID NO: 5)) in male CD-1 mice following a single intravenous dose of 50 mg/kg. To this end, mice were sacrificed at 10, 30 and minutes, and 6 and 24 hours after administration of radiolabeled AQGV (SEQ ID NO: 5), counts in various tissues were determined, and the radioactivity present in the urine and plasma were analyzed by HPLC.

There was relatively little radioactivity in the blood 10 minutes after the radiolabeled peptide was injected. If all of the counts were in intact peptide, the amount present would be 17.2 μg/g. No parent compound could be detected in plasma after 10 minutes however; [¹⁴C]-AQGV (“AQGV” disclosed as SEQ ID NO: 5) appeared to be hydrolyzed quite rapidly. No parent compound was detected in the urine either. The radioactivity in urine was mostly present as hydrophilic compounds, eluting in or just after the dead volume of the HPLC column.

The radioactivity present in various organs exceeded that in the blood. The highest concentrations of “peptide” after 10 minutes were found in the kidneys (362 μg/g), liver (105 μg/g), testis (85.7 μg/g), lung (75.2 μg/g), and spleen (74.7 μg/g). In general, a gradual decrease in radioactivity was observed thereafter. After 24 hours, the highest concentrations were found in kidneys (61.9 μg/g), thymus (43.1 μg/g), spleen (39.3 μg/g), liver (37.6 μg/g), and skin (37.5 μg/g)

The average total recoveries of radioactivity 10, 30, and 60 minutes, and 6 and 24 hours after dose administration were 83.2, 70.5, 62.9, 52.6 and 50.8%, respectively. These results strongly indicate that there is rapid formation of [14C]-volatiles after dose administration, most likely ¹⁴C—CO2, which is exhaled via the expired air. After 24 hours, 10.2% of the administered radioactivity was excreted in urine, and 2.6% in feces.

Conclusions

After intravenous injections, [¹⁴C]-AQGV (“AQGV” disclosed as SEQ ID NO: 5) was rapidly removed from the blood. This is consistent with the results of pharmacokinetic studies that are presented below. Metabolite profiles in blood plasma and urine revealed no parent compound, indicating rapid metabolism of [¹⁴C]-AQGV (“AQGV” disclosed as SEQ ID NO: 5). About 50% of the administered radioactivity was exhaled as volatiles, most likely ¹⁴C—CO2, up to 24 hours. The results of the present study indicate rapid hydrolysis of [¹⁴C]-AQGV (“AQGV” disclosed as SEQ ID NO: 5) yielding [1-¹⁴C]-glycine, which is subsequently metabolized into ¹⁴C—CO2 and exhaled in the expired air. The absence of parent compound in plasma and urine suggests that the radioactivity present in tissues and organs could be present only as hydrolyzation products of the metabolism of [¹⁴C]-AQGV (“AQGV” disclosed as SEQ ID NO: 5).

### Peptide Hydrolysis

The disclosure provides that when a peptide provide with autophagy inhibiting amino acids such as peptide AQGV (SEQ ID NO: 5) encounters a cell, the peptide is hydrolyzed, be it extracellular at the surface of that cell, or after endocytosis, in the case of vascular cells for example by elastin receptor mediated endocytosis, of the peptide by the cell in the phagolysosome. Many peptidases are known to exist on or in cells that can rapidly hydrolyze peptides, and continued hydrolysis invariably leads to tripeptides and dipeptides. Likewise, hydrolysis in the lysosomes by tripeptidyl and dipeptidyl peptidase will equally result in single amino acids. Studies with ¹⁴C labeled AQGV (SEQ ID NO: 5) have factually shown full hydrolysis of the peptide in 15 min after its administration in mice. Tripeptides, dipeptides and single amino acids will result from the hydrolysis of AQGV (SEQ ID NO: 5), or its sister compound LQGV (SEQ ID NO: 9), or for that matter from any other suitable oligopeptide, when presented to a cell.

### Peptide Transport

Similarly, several studies have reported the role of p38 MAPK in survival of different type of mature granulocytes. Granulocytes (e.g. neutrophils, eosinophils, basophils) have in common their terminal differentiation stage. These cells have fragmented nuclei and have an accumulation of granules containing preformed secretion factors. It is herein been proposed that p38 MAPK is required for survival of neutrophils, and inactivation of p38 MAPK is essential for death and the elimination of these cells as well as that p38 MAPK is required for contraction of endothelial cells, and inactivation of p38 MAPK is essential for relaxing those vascular cells so that those can restore vascular wall integrity, as well as inactivation of p38 MAPK activity is essential for pacifying neutrophils, and other leucocytes cells exploring the vascular permeability of vascular endothelial blood vessel wall.

Di- and tripeptides are selectively transported via the PEPT1/2 transporters. Tripeptides, dipeptides and single amino acids are actively transported through the cell membrane, whereby uptake of dipeptides and tripeptides involves a separate mechanism than uptake of single amino acids, namely via the PEPT1 and PEPT2 transporters. Potentially all 400 di- and 8,000 tripeptides can be transported by PepT1 and PEPT2. Intestinal cell transport of amino acids in the form of peptides was demonstrated to be a faster route of uptake per unit of time than their constituent amino acids in the free form (reviewed in J Anim Sci, 2008; 9, 2135-2155).

mTOR is Involved

Finally, we propose involvement of mechanistic target of rapamycine, mTOR. In this perspective, the peptide enters cells either via PEPT1/2 or by active endocytosing or phagocytosing processing, after which the peptide is fully hydrolyzed in the phagolysosome and the resulting autophagy inhibiting amino acids are presented to mTOR complex where they cause inhibition of autophagy of the cell. Tetrapeptide, tripeptide and dipeptide activities may all reflect the final causal activity of single amino acids A, Q, G, V, selected from the group of amino acids A, Q, G, V, L and P. In this way, the amino acids A, Q, G, V, L and P are food for mTOR. Indeed, preliminary results show similar effects on inhibition of the p38 pathway when different tri- and dipeptides derived from AQGV (SEQ ID NO: 5) in an FPR-signaling assay are used. Individual amino acids may approach mTOR via the cytosol, but amino acids in peptide fragments (strings such as AQGV (SEQ ID NO: 5)), likely enter the mTOR machinery via the phagolysosome. Activation of mTOR by amino acids can therefore be explained from two perspectives, 1) whereby endocytosis of peptide strings is paramount for all phagocytosing cells, such as neutrophils and monocytes, 2) whereby peptide fragments enter via PEPT1/2.

Amino Acids Activate mTOR Pathways and Inhibit Autophagy

Autophagy serves to produce amino acids for the survival of a cell when nutrients fall short, and amino acids are effective inhibitors of autophagy. Mechanistic-target-of-rapamicin (mTOR) is a critical regulator of autophagy induction, with activated mTOR suppressing autophagy and negative regulation of mTOR promoting it. Amino acids are indeed considered important regulators of mTOR complex 1 or 2 activation, affecting cell proliferation, protein synthesis, autophagy and survival. These findings identify new signaling pathways used by amino acids underscoring the crucial importance of these nutrients in cell metabolism and offering new mechanistic insights in developing pharmaceutically active peptides.

Differential Signaling of Amino Acids to the mTOR Pathway.

Some amino acids are known to control proteogenesis (mTOR kinases) or proteolysis (autophagy) more than others. Recent and older data (literature search November 2011) identify leucine (L), valine (V), isoleucine (I), alanine (A), glutamine (Q), arginine (R), glycine (G), proline (P), either alone or in combination, as more potent activators of mTOR or inhibitors of autophagy than other amino acids, such as glutamate (E), threonine (T), serine (S), lysine (K), threonine (T), phenylalanine (F), tyrosine (Y), and methionine (M) that have been reported to have no or opposite effects. Amino acids leucine (L), alanine (A), glutamine (Q), and proline (P) are reported to have most prominent autophagic effects on human cells (AJ Meijer et al Amino Acids 2015, 47, 2037-2063.).

TABLE

| | | | mTOR | | |
|---|---|---|---|---|---|
| Literature based activity | | | kinases | autophagy | |
| L-Glycine | Gly | G | | DOWN | Bluem J Biol Chem 2007 37783 |
| | | | | DOWN | Qin et al http://en.cnki.com.cn/Article_en/CJFDTOTAL- |
| | | | | | NJYK200912002.htm |
| L-Alanine | Ala | A | | DOWN | Proc. Natl. Acad. Sci. USA Vol. 76, No. 7, |
| | | | | | pp. 3169-3173, July 1979 |
| L-Proline | Pro | P | activate | | Washington Am J Physiol Cell Physiol 2010 298 |
| | | | | | C982 |
| L-Valine | Val | V | activate | | Maria Dolors Sans et al J. Nutr. 136: 1792-1799, 2006. |
| L-Isoleucine | Ile | I | activate | DOWN | Doi J Nutr 135 2102-8 Biochen Biophys Acta 2008 1115 |
| L-Isoleucine | | | | | Maria Dolors Sans et al J. Nutr. 136: 1792-1799, 2006. |
| L-Leucine | Leu | L | activate | DOWN | Ijichi et al Bioc Biophys Res com 2003 303 59 |
| L-Leucine | | | | | Maria Dolors Sans et al J. Nutr. 136: 1792-1799, 2006. |
| L-Glutamine | Gln | Q | activate | DOWN | Amino Acids 2009 73 111 |
| L-Glutamine | | | activate | DOWN | Kim Biol Reprod 2011 84 1139 |
| L-Arginine | Arg | R | activate | DOWN | Ban et al. Int J Mol Med 2004 13: 537-43 |
| L-Arginine | | | activate | DOWN | Kim Biol Reprod 2011 84 79 |
| L-Tyrosine | Tyr | Y | | | |
| L-Lysine | Lys | K | down | | Prizant J Cell Biochem 2008 1 1038 |
| L-Tryptophan | Trp | W | | | |
| L-Cystine | Cys | C | | | |
| L-Serine | Ser | S | | | |
| L-Threonine | Thr | T | down | | Prizant J Cell Biochem 2008 1 1038 |
| L-Asparagine | Asn | N | | | |
| L-Aspartic | Asp | D | | | |
| acid | | | | | |
| L-Methionine | Met | M | partial | | Stubs J Endocrinol 2002 174 335 |
| | | | inhibit | | |
| L-Histidine | His | H | down | | Prizant J Cell Biochem 2008 1 1038 |
| L-Phenylalanine | Phe | F | | | |
| L-Glutamic | Glu | E | | | |
| Acid | | | | | |

Peptides Enriched with Amino Acids that Inhibit Autophagy

Examples of peptides that are enriched with these above amino acids and down-regulate disease are for example di peptide AQ, QQ, LQ, GQ PQ VQ AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide AQGV (SEQ ID NO: 5), AQGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), and mixtures thereof, such as AQ+GV, and AQ+VG, and LQ+GV, and LQ+VG, which are herein provided as selected from the group of dipeptide AQ, QQ, LQ GQ PQ, VQ AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, Q0LG, QGG, QPG, QVG, LAG, LGG, LPG, LVG or a tetrapeptide AQGV (SEQ ID NO: 5), QAGV (SEQ ID NO: 18), LQGV (SEQ ID NO: 9), GQGV (SEQ ID NO: 19), PQGV (SEQ ID NO: 20), VQGV (SEQ ID NO: 21), ALGV (SEQ ID NO: 22), LLGV (SEQ ID NO: 23), QLGV (SEQ ID NO: 24), GLGV (SEQ ID NO: 25), PLGV (SEQ ID NO: 26), VLGV (SEQ ID NO: 27), QAGV (SEQ ID NO: 28), QLGV (SEQ ID NO: 24), QGGV (SEQ ID NO: 29), QPGV (SEQ ID NO: 30), QVGV (SEQ ID NO: 31), LAGV (SEQ ID NO: 32), LGGV (SEQ ID NO: 33), LPGV (SEQ ID NO: 34), LVGV (SEQ ID NO: 35), or a mixture thereof. Other peptides are now easily derived, preferably by generating or synthesizing small peptides by combining amino acids that preferentially activate mTOR or preferentially inhibit autophagy, preferably selected from the group of A, G, L, V, Q and P, into strings of peptides.

### Assumed Mode of Action

Administered peptide or amino acid fragments thereof are for example taken up by amino acid transport, PEPT1/2 transport, by common endocytosis, in the case of vascular cells by elastin receptor mediated endocytosis or by common phagocytosis. Internalized peptide is hydrolyzed and its amino acids are presented to the nutrient-sensing system of mTOR. As it now herein emerges, these peptides preferably need be hydrolyzed into individual amino acids before they can act at the nutrient-sensing-system of mTOR, thus it can be understood why receptor meditated activity has never unequivocally been demonstrated. As to routing into the cell, most di- and tripeptides are readily taken up by PEPT1/2 transporters present in intestinal epithelial cells, renal tubular cells and other cells. Also, tetra- to hexapeptide uptake is regularly achieved by common endocytosis, in the case of vascular cells by elastin receptor mediated endocytosis, allowing targeting cells for uptake by phagocytosis. Internalized peptide is hydrolyzed and its amino acids are presented to the nutrient-sensing system of mTOR. Considering the broad mode-of-action here displayed, the tissue-repair signal molecule peptides provided in the disclosure can advantageously be used in combined treatment with many biologic therapies

### Rational Design

A great advantage of this new class of autophagy inhibiting peptides (herein also indicated as autophagy inhibiting molecules) is that they are easily synthesized, stabilized and modified, the main requirement being that they comprise amino acids that target the nutrient sensing system of mTOR and preferentially inhibit autophagy.

### Human Trials

### Pharmacokinetics, Safety and Tolerability of the Novel Compound AQGV (SEQ ID NO: 5)

### Background and Objective

Having performed extensive research into the pharmacology, pharmacokinetics and toxicology of AQGV (SEQ ID NO: 5), a first in human study was previously conducted with escalating single doses of AQGV (SEQ ID NO: 5), which showed that AQGV (SEQ ID NO: 5) was well tolerated up to i.v. doses of 30 mg/kg three times a day (daily dose of 90 mg/kg) for three days, and did not result in adverse events that were related to the study treatment.

The objectives of this phase I study were to evaluate the pharmacokinetics (PK), safety and tolerability of AQGV (SEQ ID NO: 5) in humans.

Methods

Three double-blind, randomized, placebo-controlled, dose-escalating phase I studies in healthy subjects were conducted to evaluate safety, tolerability and pharmacokinetics of AQGV (SEQ ID NO: 5). In the first single dose study, 32 subjects were assigned to four single dosage groups (1, 3, 10 or 30 mg/kg). In the second multiple dosage study, 24 subjects were assigned to three dosing groups (10, 20 or 30 mg/kg) and received study medication thrice a day for three days. In the third continuous dosing study, 24 subjects were assigned to three dosing groups (15, 30 or 90 mg/kg/hour) to receive study medication by 2-hour continuous iv infusion. Pharmacokinetics (PK), safety and tolerability assessments were performed up to 14-days.

Results

PK of AQGV (SEQ ID NO: 5) showed more than proportional increases in exposure to the highest dosages (range: 126-137%), a large volume of distribution (range: 4-33 L/kg), a fast clearance rate (range: 26-61 L/h/kg) and a short estimated half life time (range: 2-22 minutes). AQGV (SEQ ID NO: 5) was well tolerated and no safety concerns were observed; no serious adverse events (SAES) or discontinuation of administration due to adverse events (AEs) were reported. All but one AE were mild and only 6 subjects reported AEs with a possible relationship to the study drug treatment, by 3 subjects receiving AQGV (SEQ ID NO: 5) and by 3 subjects receiving placebo. All other experienced AEs were considered not or unlikely to study drug treatment.

Conclusion

These dose-escalating phase I studies with different administration strategies reveal a PK profile of AQGV (SEQ ID NO: 5) with a large volume of distribution and a short half-life time. Furthermore, i.v. administration of AQGV (SEQ ID NO: 5) was well tolerated and no safety issues emerged.

### Solubility Studies

### Peptide Administration

A shown in clinical trial protocol (Groenendael et al., JMIR Res Protoc 2019 February; 8(2): e11441.), study medication EA-230 formulation is packed and provided in sterile 5-mL glass vials, containing 1500 mg/vial, dissolved in water for injection at a final concentration of 300 mg/mL with an osmolality of 800 to 1000 mOsm/kg. The placebo formulation consists of sodium chloride diluted in water for injection in identical sterile 5-mL glass vials containing 29 mg/mL to reach a solution with an identical osmolality. EA-230 and placebo are prepared for continuous intravenous infusion with an osmolality of <400 mOsm/kg by adding the appropriate amount of EA-230 or placebo to 1000 mL normal saline under aseptic conditions.

Need for Stock-Solution with Higher Concentration Active Substance.

A vial with EA-230 formulation (stock-solution) used in herein referenced clinical trial contained 1.5 gram EA-230, each vial containing 5 ml a 300 mg/mI[(300 g/L=0.8 mol/L) AQGV (SEQ ID NO: 5) having a molecular weight of 373 g/mol]. In said trial, a best-treatment practice was established when infusion with active substance lasted at least 1.5 hours, preferably at least 2.5 hours, preferably at least, 3.5 hours, more preferably at least 4.5 hours, at 90 mg/kg per hour. As a consequence, and also depending on bodyweight, often more than 12-17 vials were needed for continuing effective treatment, an administration requirement that takes (too) much labor in the operating room or ICU for the required care. This disadvantage of treatment with too weak amounts of stock of EA-230 formulation brings forward a need to provide more and better concentrated stock-solutions than available.

### Determination of Aggregation Points

It is recognized herein that many drug-like molecules can self-aggregate in aqueous media and aggregates may have physicochemical properties that skew experimental results and clinical decisions. The aggregation of peptide drugs is one of the most common and troubling processes encountered in almost all phases of biological drug development. Aggregation can take several different forms and the term is used to describe a number of different processes during which peptide molecules associate into larger species consisting of multiple polypeptide chains. Aggregates can be amorphous or highly structured, e.g. amyloid fibrils, and can form in solution or on surfaces due to adsorption. They can arise as a result of the non-covalent association of polypeptide chains, or from covalent linkage of chains. In some cases, aggregation is reversible while in others it is effectively irreversible. In either case, it reduces the physical stability of the peptide in question, not only leading to a loss in activity but also other critical problems such as toxicity and immunogenicity.

Salts have complex effects on the physical stability of biomolecules affecting both conformational and colloidal stability. Their effects frequently vary according to the surface charge on the peptide and the overall effect of a salt on physical stability is a balance of different and multiple mechanisms by which salt interacts with water and biomolecules. Various salts can influence physical stability by altering the properties of the peptide-solvent system (Hofmeister effects) and by altering electrostatic interactions (Debye-Hückel effects).

We aimed to investigate the solubility of seven different salts on prototype autophagy inhibiting peptide AQGV (SEQ ID NO: 5), using the modified shake flask method. At first the AQGV-Ac salt (“AQGV” disclosed as SEQ ID NO: 5) will be converted to the free base, extracted with an organic solvent and concentrated in vacua. Subsequently the citrate, maleate, sulfate (KHSO4), adenosine mono-phosphate, adenosine, acetate and tartaric acid salts will be prepared and screened for their solubility thereafter.

Results

Conversion to the Free Base.

Extraction of AQGV-Ac (“AQGV” disclosed as SEQ ID NO: 5) with organized solvents from neutralized solution (pH=6-7) turned out not to be possible. Therefore a solution of AQGV-Ac (“AQGV” disclosed as SEQ ID NO: 5) in water was transferred to an ion-exchange column (Amberlite, approximately 100 mL; IR120, H resin) the column was flushed using demi-water followed by 1N ammonia-solution. The first 3 basic fractions were concentrated to afford 4.7 g of the free base AQGV (SEQ ID NO: 5) (1H-NMR).

### Solubility Measurements.

At first attempts were made to mix a solution of the free base and an acid in order to achieve a concentrated DMSO solution of the salt and subsequently dilute this in water in order to determine solubility. However the salts attempted (adenosine and citric acid) did not dissolve in DMSO at all. In fact, the mixture became clear after the addition of a little water. Therefore the solubility determinations could not be conducted as planned originally. It was decided to determine the solubility of the salts required by dilution of known amount of salts (not soluble) till a clear solution is obtained.

For citric acid, 1 mmol AQGV (SEQ ID NO: 5) and 1 mmol citric acid were mixed in 0.5 mL 0.9% NaCl. This afforded a clear solution. More material of both AQGV (SEQ ID NO: 5) and citric acid were added (amounts of 0.5 and 0.25 mmol) until a total of 2.75 mmol was dissolved in the 0.5 mL 0.9% NaCl. The mixture remains clear but got very thick/viscous. The remaining experiments have been conducted differently: 1 or 0.5 mmol salt was weighed in a 4 ml vial and small amounts of 0.9% NaCl were added until a clear solution was obtained, which remained clear for more than a week. In case of adenosine and adenosine-monophosphate no clear solution could be obtained.

Based on the results depicted in Table 1 the concentration below which an aggregated peptide-salt tends to resolve of neutral and autophagy inhibiting peptide-salts screened were determined (aggregation points, see Table 2). It can be concluded that changing the anion significantly influences the solubility characteristics of AQGV (SEQ ID NO: 5). Higher solubility (solubility in 0.9% NaCl) and therewith higher aggregation points were observed for the AQGV-citric acid (“AQGV” disclosed as SEQ ID NO: 5) (AQGV-citrate (“AQGV” disclosed as SEQ ID NO: 5), >5.5 mol/L) and -tartaric acid (AQGV-tartrate (“AQGV” disclosed as SEQ ID NO: 5)) salt, whereas maleic acid and KHSO4 salts showed lower solubility, compared to AQGV-Ac (“AQGV” disclosed as SEQ ID NO: 5) (2 mol/L). Using adenosine-monophosphate or adenosine in our hands did not provide solubility. Citric acid seems to be a special case. Highly concentrated solution does not crystallize or aggregate but tend to form a highly viscous solution.

Heeding aggregation risk, a vial with a stock solution of AQGV-peptide (SEQ ID NO: 5) for use in a clinical trial hitherto contained no more than (0.8 mol/L) active substrate in solution. Based on the current invention such a stock solution of an AQGV-salt (SEQ ID NO: 5) of an organic acid, in particular of AQGV-peptide-maleate (“AQGV” disclosed as SEQ ID NO: 5), AQGV-peptide-acetate (“AQGV” disclosed as SEQ ID NO: 5) AQGV-peptide-tartrate (“AQGV” disclosed as SEQ ID NO: 5) or AQGV-peptide-citrate (“AQGV” disclosed as SEQ ID NO: 5) now is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of said AQGV-peptide-acetate (“AQGV” disclosed as SEQ ID NO: 5), AQGV-peptide tartrate (“AQGV” disclosed as SEQ ID NO: 5) or AQGV-peptide-citrate (“AQGV” disclosed as SEQ ID NO: 5). In a more preferred embodiment, the invention provides a stock-solution of said AQGV-peptide-tartrate (“AQGV” disclosed as SEQ ID NO: 5) or said AQGV-peptide-citrate (“AQGV” disclosed as SEQ ID NO: 5) wherein the concentration of said AQGV-peptide (SEQ ID NO: 5) is in the range of 2 mol/L to 2.5 mol/L. In a more preferred embodiment, the invention provides a stock-solution of said AQGV-peptide-citrate (“AQGV” disclosed as SEQ ID NO: 5) wherein the concentration of said peptide-citrate is in the range of 2.5 mol/L to 3 mol/L. In a more preferred embodiment, the invention provides a stock-solution of said peptide-citrate wherein the concentration of said peptide-citrate is in the range of 3 mol/L to 3.5 mol/L. In a more preferred embodiment, the invention provides a stock-solution of said peptide-citrate wherein the concentration of said peptide-citrate is in the range of 3.5 mol/L to 4.5 mol/L. In a more preferred embodiment, the invention provides a stock-solution of said peptide-citrate wherein the concentration of said peptide-citrate is in the range of 4.5 mol/L to 5.5 mol/L. In a more preferred embodiment, the invention provides a stock-solution of said peptide-citrate wherein the concentration of said peptide citrate is equal to or larger than 5.5 mol/L. It is preferred that said stock solution is an aqueous solution.

Tartaric acid, also known as 2,3-dihydroxybutanedioic acid, is a diprotic aldaric acid. It occurs naturally in many plants, particularly grapes, bananas, and tamarinds, is commonly combined with baking soda to function as a leavening agent in recipes, and is one of the main acids found in wine. It is added to other foods to give a sour taste, and is used as an antioxidant. A tartrate is a salt or ester of tartaric acid. A salt of tartaric acid is known as a tartrate. Tartaric acid is a dihydroxyl derivative of succinic acid. Sodium tartrate is a disodium salt of (+)-tartaric acid that occurs as transparent, colorless, and odorless crystals. It is obtained as a byproduct of wine manufacture. Sodium tartrate is generally recognized as safe (GRAS) as a direct human food ingredient. It acts as an emulsifier and pH control agent in food products. It is known to help increase oral absorption of metopronol (Am J Kidney Dis. 2014 December; 64(6):883-91) and ergotamine (Can Med Assoc J. 1935 December; 33(6):664-5). Protein compositions comprising tartrate as a pharmaceutical excipient are reported to provide stability (U.S. Ser. No. 07/716,390) to pharmaceutical preparations for nasal application. Recent data reveal that polymorphic forms of both citrate and tartrate salt of sildenafil are the same, indicating that tartrate could be used as an alternate excipient to citrate (Research J. Pharm. and Tech 2018; 11(5):2086-2093. doi: 10.5958/0974-360X.2018.00387.6).

Citric acid, also known as 2-hydroxypropane-1,2,3-tricarboxylic acid, is a tricarboxylic acid found in citrus fruits. Citric acid is often used as an excipient in pharmaceutical preparations due to its antioxidant properties. It maintains stability of active ingredients and is used as a preservative. It is also used as an acidulant to control pH and acts as an anticoagulant by chelating calcium in blood. A citrate is a salt or ester of citric acid. A salt of citric acid is a citrate. Upon absorption, sodium citrate dissociates into sodium cations and citrate anions; organic citrate ions are metabolized to bicarbonate ions, resulting in an increase in the plasma bicarbonate concentration, the buffering of excess hydrogen ion, the raising of blood pH, and potentially the reversal of acidosis. In addition, increases in free sodium load due to sodium citrate administration may increase intravascular blood volume, facilitating the excretion of bicarbonate compounds and an anti-urolithic effect. Citrate is used as an anticoagulant during plasmophoresis as well as a neutralizing agent in the treatment of upset stomach and acidic urine. Sodium citrate is generally recognized as safe (GRAS) as a direct human food ingredient. Citrate is used in critically ill patients undergoing citrate-anticoagulated continuous venovenous haemofiltration (CVVH). As pharmaceutical excipient, citric acid has been reported to improve oral absorption of small molecules and proteins by different mechanisms. The balance between its related properties of calcium chelation and intestinal permeation enhancement compared to a proteolysis inhibition was examined earlier with insulin (Eur J Pharm Biopharm. 2014 April; 86(3):544-51). Citrate is also known to help increase oral absorption of sildenafil (Int J Impot Res. 1996 June; 8(2):47-52) and is included as butamirate citrate in cough syrup (Rev Med Suisse Romande. 990 November; 110(11):983-6).

### FPR Mediated Vascular Permeability and Hypotension

Although the concept that active contraction of endothelial cells regulate vascular permeability was first suggested by Majno in 1961 (J Biophys Biochem Cytol (1961) 11:571.10.1083/jcb.11.3.571), currently the intracellular events regulating endothelial contractile activity are still relatively unknown. N-Formyl peptides are common molecular signatures of bacteria and mitochondria that activate the formyl peptide receptor (FPR). FPR activation by mitochondrial N-formyl peptides (F-MIT) or by bacterial N-formyl peptides (F-MLP) such as N-formyl-methionyl-leucyl-phenylalanine elicits changes in cytoskeleton-regulating proteins in endothelial cells that lead to increased endothelial cell contractility with increased vascular leakage and extravasation of leukocytes. FPR activation is a key contributor to impaired barrier function in following trauma. It has been proposed that in patients, mitochondrial components from damaged tissue can initiate the genesis of vascular leakage (Wenceslau et al., Front Immunol. 2016; 7: 297). For evolutionary reasons, mitochondria share several characteristics with bacteria, and when fragments of mitochondria are released into the circulation, they are recognized by cells carrying the formyl-peptide-receptor (FPR). Due to protein translation initiation by formyl-methionine in both bacteria and mitochondria, N-formyl peptides are common molecular signatures of bacteria and mitochondria and are known to play a role in the initiation of vascular leakage by activating the formyl peptide receptor (FPR).

The FPR has been identified as a subfamily of G-protein-coupled receptors. Recent evidence also suggests that FPR is a membrane mechanosensor that senses the mechanical fluid stress in of our vascular system and signals intracellular cascades. It has also been observed that both mitochondrial N-formyl peptides (formylated peptide corresponding to the NH₂-terminus of mitochondria NADPH dehydrogenase subunit 6; F-MIT) and fMLP (bacteria derived) induce vascular leakage and exacerbated vasodilatation in resistance arteries, and that a FPR antagonist inhibits these responses. F-MIT, but not non-formylated peptides or mitochondrial DNA, also induced severe hypotension via FPR activation and histamine release (Wenceslau et al., Am J Physiol Heart Circ Physiol. 2015 Apr. 1; 308(7):H768-77). Mitogen-activated protein (MAP) kinases are a family of stress activated enzymes that initiate signaling cascades in response to several stimuli, including injury. It has previously been shown that p38 MAPK kinase leads to reorganization of the actin cytoskeleton to form stress fibers and increase in vascular permeability (Kayyali et al., J Biol Chem (2002) 277(45): 42596-602.10.1074/jbc.M205863200). Furthermore, it was recently shown that inhibition of the p38 MAPK pathway ameliorates resulting vascular dysfunction by significantly reducing endothelial cells contraction (Wang et al, APMIS (2014) 122(9):832-41.10.1111/apm.12226). In conclusion, mitochondrial N-formyl peptides (F-MIT) released from trauma/cell damage activate formyl peptide receptor (FPR). Those events lead to p38 MAPK kinase activated changes in endothelial cell cytoskeleton which subsequently induces endothelial contraction and therewith induces detrimental vascular effects such as vascular permeability with edema, vascular leakage, adverse leukocyte extravasation and hypotension.

### Autophagy Inhibiting Peptide Modulate FPR-Permeability and Vascular Leakage Via Inhibiting Cytoskeleton Changes.

We herein disclose that autophagy inhibiting peptides such as AQGV (SEQ ID NO: 5) and functional analogues, modulate p38 MAPK kinase activated changes in endothelial cell cytoskeleton and subsequently modulate endothelial contraction and therewith improve vascular permeability, leukocyte extravasation and hypotension. Mitogen-activated protein (MAP) kinases are a family of stress activated enzymes that initiate signaling cascades in response to several stimuli, including injury. As previously shown, p38 MAPK kinase leads to reorganization of the actin cytoskeleton in endothelial cells to form stress fibers and increases in vascular permeability (Kayyali et al., J Biol Chem (2002) 277(45):42596-602.10.1074/jbc.M205863200). Furthermore, it is thought that damaged or stressed endothelial cells activate said acute reorganization of the actin cytoskeleton in endothelial cells through p38 signaling effects on the PI3K/Akt/mTOR pathway. This acute response to endothelial cell stress occurs in the context of PI3K/AKT/mTOR pathway down-regulation and upregulation of autophagy, to shift balance from proteogenesis to proteolysis in said cells. As also recently shown, inhibition of the p38 MAPK pathway ameliorates resulting vascular dysfunction by significantly reducing endothelial cells contraction (Wang et al, APMIS (2014) 122(9):832-41.10.1111/apm.12226).

Human-derived peripheral blood monocytes as a prototype cell-system for FPR-expression were isolated from healthy volunteers (hPBMCs) according to routine procedures¹ at University Medical Centre Groningen, The Netherlands. Subsequently PMBC were incubated with the Biotempt-supplied peptide AQGV (SEQ ID NO: 5) (which was freshly prepared as a 20 meml stock solution in bidistilled water) or treated with the vehicle for 10 min. Then hPMBCs were challenged with 1 μM fMLP for various time periods (see results FIGS. 4a, b, 4c and 4d). Each stimulus 0, 30, 60, 300 and 600 seconds at two concentrations AQGV (SEQ ID NO: 5), 20 ng/ml and 50 ng/ml and in six-fold. Following stimulation with fMLP for the indicated time periods, the phosphorylation status of p38MAPK, JNK, PKB, P42/p44MAPK/ERK1,2 and STAT3 was analysed 3. STAT3 was undetectable, JNK and P42/p44MAPK/ERK1,2.

Results (see also FIG. 4) show that FPR-activation of FPR-expressing cells with prototype FPR-ligand fMLP causes rapidly induced and significant (p<0.05; p38 from 60 to 600 sec, PKB at 600 sec) changes in phosphorylation status of PKB (also known as AKT) (FIG. 4a) and p38 MAPK kinases (FIG. 4c), but not (or not detected) in STAT3, JNK (FIG. 4b) and P42/p44MAPK/ERK1,2 (FIG. 4d) kinases. AQGV (SEQ ID NO: 5) effects on p38 MAPK (FIG. 4c) are already detected at seconds after FPR-stimulation, AQGV (SEQ ID NO: 5) effects on PKB(AKT) follow (FIG. 4a) in a bi-phasic pattern at 300 sec. Both AQGV (SEQ ID NO: 5) effects on p38 and PKB-mediated signalling last for the full 600 seconds tested whereas the other kinases tested were not affected throughout. This acute and specific response to treatment shows specific and rapid effects of autophagy-inhibiting-AQGV-peptide (SEQ ID NO: 5) on p38 signaling in the context of regulation of the PI3K/AKT/mTOR pathway. Said pathway is governing the balance between proteolysis and proteogenesis regulating cytoskeleton changes affecting vascular permeability. It is shown that AQGV-peptide (SEQ ID NO: 5) reduces p38 MAPK kinase activated changes as well as reduces PI3K/AKT/mTOR activated induced changes in cell cytoskeleton reorganization affecting endothelial cell contraction and adverse vascular permeability, AQGV-peptide (SEQ ID NO: 5) is useful and capable of addressing adverse vascular permeability, such as manifested by edema with vascular leakage, adverse leukocyte extravasation and hypotension in human subjects.

### REFERENCES

¹van den Blink B, Branger J, Weijer S, Deventer S H, van der Poll T, Peppelenbosch M P. Human endotoxemia activates p38 MAP kinase and p42/44 MAP kinase, but not c-Jun N-terminal kinase. Mol Med. 2001 November; 7(11):755-60.
²O'Toole T, Peppelenbosch M P. Phosphatidyl inositol-3-phosphate kinase mediates CD14 dependent signaling. Mol Immunol. 2007 March; 44(9):2362-9.
³Versteeg H H, Nijhuis E, van den Brink G R, Evertzen M, Pynaert G N, van Deventer S J, Coffer P J, Peppelenbosch M P. A new phosphospecific cell-based ELISA for p42/p44 mitogen-activated protein kinase (MAPK), p38 MAPK, protein kinase B and cAMP-response-element-binding protein. Biochem J. 2000 Sep. 15; 350:717-22.
⁴Bos C L, Diks S H, Hardwick J C, Walburg K V, Peppelenbosch M P, Richel D J. Protein phosphatase 2A is required for mesalazine-dependent inhibition of Wnt/beta-catenin pathway activity. Carcinogenesis. 2006 27:2371-82

### Example 1

AQGVLPGQ-maleate (“AQGVLPGQ” disclosed as SEQ ID NO: 1)

To prepare 1 L of the composition, mix
AQGVLPGQ-maleate (“AQGVLPGQ” disclosed as SEQ ID NO: 1)—1.8 mol
0.9% NaCl—1 L

### Example 2

LQGVLPGQ-maleate (“LQGVLPGQ” disclosed as SEQ ID NO: 2)

To prepare 1 L of the composition, mix
LQGVLPGQ-maleate (“LQGVLPGQ” disclosed as SEQ ID NO: 2)—1.8 mol
0.9% NaCl—1 L

### Example 3

AQGLQPGQ-maleate (“AQGLQPGQ” disclosed as SEQ ID NO: 3)

To prepare 1 L of the composition, mix
AQGLQPGQ-maleate (“AQGLQPGQ” disclosed as SEQ ID NO: 3)—1.8 mol
NaCl—1 L

### Example 4

LQGLQPGQ-maleate (“LQGLQPGQ” disclosed as SEQ ID NO: 4)

To prepare 1 L of the composition, mix
LQGLQPGQ-maleate (“LQGLQPGQ” disclosed as SEQ ID NO: 4)—1.8 mol
NaCl—1 L

### Example 5

AQGV-maleate (“AQGV” disclosed as SEQ ID NO: 5)

To prepare 1 L of the composition, mix
AQGV-maleate (“AQGV” disclosed as SEQ ID NO: 5)—1.8 mol
NaCl—1 L

### Example 6

LQGVL-maleate (“LQGVL” disclosed as SEQ ID NO: 6)

To prepare 1 L of the composition, mix
LQGVL-maleate (“LQGVL” disclosed as SEQ ID NO: 6)—1.8 mol
NaCl—1 L

### Example 7

AQGLQ-maleate (“AQGLQ” disclosed as SEQ ID NO: 7)

To prepare 1 L of the composition, mix
AQGLQ-maleate (“AQGLQ” disclosed as SEQ ID NO: 7)—1.8 mol
NaCl—1 L

### Example 8

LQGLQ-maleate (“LQGLQ” disclosed as SEQ ID NO: 8)

To prepare 1 L of the composition, mix
LQGLQ-maleate (“LQGLQ” disclosed as SEQ ID NO: 8)—1.8 mol
0.9% NaCl—1 L

### Example 9

AQGVLPGQ-acetate (“AQGVLPGQ” disclosed as SEQ ID NO: 1)

To prepare 1 L of the composition, mix
AQGVLPGQ-acetate (“AQGVLPGQ” disclosed as SEQ ID NO: 1)—1.8 mol
0.9% NaCl—1 L

### Example 10

LQGVLPGQ-acetate (“LQGVLPGQ” disclosed as SEQ ID NO: 2)

To prepare 1 L of the composition, mix
LQGVLPGQ-acetate (“LQGVLPGQ” disclosed as SEQ ID NO: 2)—1.8 mol
NaCl—1 L

### Example 11

AQGLQPGQ-acetate (“AQGLQPGQ” disclosed as SEQ ID NO: 3)

To prepare 1 L of the composition, mix
AQGLQPGQ-acetate (“AQGLQPGQ” disclosed as SEQ ID NO: 3)—1.8 mol
NaCl—1 L

### Example 12

LQGLQPGQ-acetate (“LQGLQPGQ” disclosed as SEQ ID NO: 4)

To prepare 1 L of the composition, mix
LQGLQPGQ-acetate (“LQGLQPGQ” disclosed as SEQ ID NO: 4)—1.8 mol
NaCl—1 L

### Example 13

AQGV-acetate (“AQGV” disclosed as SEQ ID NO: 5)

To prepare 1 L of the composition, mix
AQGV-acetate (“AQGV” disclosed as SEQ ID NO: 5)—1.8 mol
0.9% NaCl—1 L

### Example 14

LQGVL-acetate (“LQGVL” disclosed as SEQ ID NO: 6)

To prepare 1 L of the composition, mix
LQGVL-acetate (“LQGVL” disclosed as SEQ ID NO: 6)—1.8 mol
0.9% NaCl—1 L

### Example 15

AQGLQ-acetate (“AQGLQ” disclosed as SEQ ID NO: 7)

To prepare 1 L of the composition, mix
AQGLQPGQ-acetate (“AQGLQPGQ” disclosed as SEQ ID NO: 3)—1.8 mol
0.9% NaCl—1 L

### Example 16

LQGLQ-acetate (“LQGLQ” disclosed as SEQ ID NO: 8)

To prepare 1 L of the composition, mix
LQGLQ-acetate (“LQGLQ” disclosed as SEQ ID NO: 8)—1.8 mol
NaCl—1 L

### Example 17

AQGVLPGQ-tartrate (“AQGVLPGQ” disclosed as SEQ ID NO: 1)

To prepare 1 L of the composition, mix
AQGVLPGQ-tartrate (“AQGVLPGQ” disclosed as SEQ ID NO: 1)—1.8 mol
NaCl—1 L

### Example 18

LQGVLPGQ-tartrate (“LQGVLPGQ” disclosed as SEQ ID NO: 2)

To prepare 1 L of the composition, mix
LQGVLPGQ-tartrate (“LQGVLPGQ” disclosed as SEQ ID NO: 2)—1.8 mol
NaCl—1 L

### Example 19

AQGLQPGQ-tartrate (“AQGLQPGQ” disclosed as SEQ ID NO: 3)

To prepare 1 L of the composition, mix
AQGLQPGQ-tartrate (“AQGLQPGQ” disclosed as SEQ ID NO: 3)—1.8 mol
NaCl—1 L

### Example 20

LQGLQPGQ-tartrate (“LQGLQPGQ” disclosed as SEQ ID NO: 4)

To prepare 1 L of the composition, mix
LQGLQPGQ-tartrate (“LQGLQPGQ” disclosed as SEQ ID NO: 4)—1.8 mol
NaCl—1 L

### Example 21

AQGV-tartrate (“AQGV” disclosed as SEQ ID NO: 5)

To prepare 1 L of the composition, mix
AQGV-tartrate (“AQGV” disclosed as SEQ ID NO: 5)—1.8 mol
NaCl—1 L

### Example 22

LQGVL-tartrate (“LQGVL” disclosed as SEQ ID NO: 6)

To prepare 1 L of the composition, mix
LQGVL-tartrate (“LQGVL” disclosed as SEQ ID NO: 6)—1.8 mol
NaCl—1 L

### Example 23

AQGLQ-tartrate (“AQGLQ” disclosed as SEQ ID NO: 7)

To prepare 1 L of the composition, mix
AQGLQ-tartrate (“AQGLQ” disclosed as SEQ ID NO: 7)—1.8 mol
NaCl—1 L

### Example 24

LQGLQ-tartrate (“LQGLQ” disclosed as SEQ ID NO: 8)

To prepare 1 L of the composition, mix
LQGLQ-tartrate (“LQGLQ” disclosed as SEQ ID NO: 8)—1.8 mol
NaCl—1 L

### Example 25

AQGVLPGQ-citrate (“AQGVLPGQ” disclosed as SEQ ID NO: 1)

To prepare 1 L of the composition, mix
AQGVLPGQ-citrate (“AQGVLPGQ” disclosed as SEQ ID NO: 1)—1.8 mol
0.9% NaCl—1 L

### Example 26

LQGVLPGQ-citrate (“LQGVLPGQ” disclosed as SEQ ID NO: 2)

To prepare 1 L of the composition, mix
LQGVLPGQ-citrate (“LQGVLPGQ” disclosed as SEQ ID NO: 2)—1.8 mol
NaCl—1 L

### Example 27

AQGLQPGQ-citrate (“AQGLQPGQ” disclosed as SEQ ID NO: 3)

To prepare 1 L of the composition, mix
AQGLQPGQ-citrate (“AQGLQPGQ” disclosed as SEQ ID NO: 3)—1.8 mol
NaCl—1 L

### Example 28

LQGLQPGQ-citrate (“LQGLQPGQ” disclosed as SEQ ID NO: 4)

To prepare 1 L of the composition, mix
LQGLQPGQ-citrate (“LQGLQPGQ” disclosed as SEQ ID NO: 4)—1.8 mol
NaCl—1 L

### Example 29

AQGV-citrate (“AQGV” disclosed as SEQ ID NO: 5)

To prepare 1 L of the composition, mix
AQGV-citrate (“AQGV” disclosed as SEQ ID NO: 5)—1.8 mol
NaCl—1 L

### Example 30

LQGVL-citrate (“LQGVL” disclosed as SEQ ID NO: 6)

To prepare 1 L of the composition, mix
LQGVL-citrate (“LQGVL” disclosed as SEQ ID NO: 6)—1.8 mol
NaCl—1 L

### Example 31

AQGLQ-citrate (“AQGLQ” disclosed as SEQ ID NO: 7)

To prepare 1 L of the composition, mix
AQGLQ-citrate (“AQGLQ” disclosed as SEQ ID NO: 7)—1.8 mol
NaCl—1 L

### Example 32

LQGLQ-citrate (“LQGLQ” disclosed as SEQ ID NO: 8)

To prepare 1 L of the composition, mix
LQGLQ-citrate (“LQGLQ” disclosed as SEQ ID NO: 8)—1.8 mol
NaCl—1 L

1. A method for reducing p38 MAPK kinase activity, for reducing PI3K/AKT/mTOR activity, or for reducing cytoskeleton reorganization, comprising providing cells with a source of autophagy inhibiting amino acids, wherein at least 50% of said amino acids are selected from the group of alanine (in one letter code: A), glutamine (Q), glycine (G), valine (V), leucine (L), isoleucine (I), proline (P) and arginine (R).

2. (canceled)

3. (canceled)

4. A method for identifying a peptide capable of reducing p38 MAPK kinase activity, for identifying a peptide capable of reducing PI3K/AKT/mTOR activity, or for identifying a peptide capable of reducing cytoskeleton reorganization, comprising providing cells with a peptide consisting of amino acids, wherein at least 50% of said amino acids are selected from the group of alanine (in one letter code: A), glutamine (Q), glycine (G), valine (V), leucine (L), isoleucine (I), proline (P) and arginine (R), providing said cells with fMLP and detecting phosphorylation of p38 MAPK in the absence and presence of said peptide at 30 to 600 seconds after provision of fMLP, and comparing the results to determine said peptide's effect on said phosphorylation.

5. (canceled)

6. (canceled)

7. The method according to claim 1 wherein at least 75% of said amino acids are for selected from said group of autophagy inhibiting amino acids.

8. The method according to claim 1 wherein said amino acids are selected from said group of autophagy inhibiting amino acids.

9. The method for identifying a peptide according to claim 4 wherein said peptide is provided as a salt of an organic acid.

10. The method for identifying a peptide according to claim 4 wherein said peptide is provided as a salt of an organic acid selected from the group of maleic acid, acetic acid, tartaric acid and citric acid.

11. The method for identifying a peptide according to claim 4 wherein said peptide is provided as a salt of of citric acid.

12. A stock solution comprising a source of autophagy inhibiting amino acids, said amino acids are for at least 50% selected from the group of alanine (in one letter code: A), glutamine (Q), glycine (G), valine (V), leucine (L), isoleucine (I), proline (P) and arginine (R).

13. The stock solution according to claim 12, wherein said amino acids are for at least 75% selected from said group of autophagy inhibiting amino acids.

14. The stock solution according to claim 12, wherein said amino acids are selected from said group of autophagy inhibiting amino acids.

15. A stock solution comprising a source of autophagy inhibiting amino acids, said amino acids are for at least 50% selected from the group of alanine (in one letter code: A), glutamine (Q), glycine (G), valine (V), leucine (L), isoleucine (I), proline (P) and arginine (R), wherein said source comprises at least one peptide comprising said at least 50% amino acids, wherein said peptide is present as a salt of an organic acid.

16. The stock solution according to claim 15, comprising at least a peptide wherein said amino acids are for at least 75% selected from said group of autophagy inhibiting amino acids.

17. The stock solution according to claim 12 for use in a method for reducing p38 MAPK kinase activity, for reducing PI3K/AKT/mTOR activity, for reducing cytoskeleton reorganization, for identifying a peptide capable of reducing PI3K/AKT/mTOR activity, or for identifying a peptide capable of reducing cytoskeleton reorganization.

18. An aqueous solution useful in fluid resuscitation and/or addressing issues of vascular permeability of a patient prepared with a stock solution according to claim 15.

19. The aqueous solution according to claim 18 wherein said patient is a human.

20. The aqueous solution according to claim 19 wherein said patient is considered critically ill.

21. The aqueous solution according to claim 18, for use in treatment of a disease associated with increased vascular permeability.

22. The solution according to claim 18 for use in treatment of increased vascular permeability.

23. The solution according to claim 18 for use in prevention or treatment of fluid overload.

24. The solution according to claim 18 that is a resuscitation fluid.

25. The solution according to claim 18 that is a crystalloid.

26. The A method according to claim 4 wherein at least 75% of said amino acids are selected from said group of autophagy inhibiting amino acids.

27. The method according to claim 4 wherein said amino acids are selected from said group of autophagy inhibiting amino acids.

28. The stock solution according to 15 wherein the salt of an organic acid is a salt of maleic acid, a salt of tartaric acid, or a salt of citric acid.
